(19) United States
(12) Patent Application Publication
(10) Pub. No.: US 2022/0152264 A1
(43) Pub. Date: May 19, 2022

(54) CONTROL OF A MICROWAVE ENHANCED AIR DISINFECTION SYSTEM

(71) Applicant: Vektra Systems LLC, Sacramento, CA (US)

(72) Inventors: Chang Yul Cha, Roseville, CA (US); Suk-Bae Cha, Tokyo (JP); George Crandell, Sacramento, CA (US); Craig Henricksen, Oakland, CA (US); William Walden, Fair Oaks, CA (US)

(21) Appl. No.: 17/522,689

(22) Filed: Nov. 9, 2021

Related U.S. Application Data
(60) Provisional application No. 63/113,690, filed on Nov. 13, 2020.

(51) Int. Cl. A61L 9/18; G05B 13/02

(52) U.S. Cl. CPC A61L 9/18; A61L 2209/14; A61L 2209/111; G05B 13/0265

(57) ABSTRACT

A method includes identifying a schedule to operate a microwave enhanced air disinfection (MEAD) system and causing, based on the schedule, intermittent generation of microwave energy by a microwave generator of the MEAD system. A multi-component filter disposed in a housing of the MEAD system is configured to collect contaminants from airflow through the housing. At least a portion of the contaminants from the airflow is to be destroyed at least one of directly or indirectly via the microwave energy.

### RELATED APPLICATION

This application claims benefit of Provisional Application No. 63/113,690, filed Nov. 13, 2020, the entire content of which is incorporated by reference herein.

### TECHNICAL FIELD

Embodiments of the present disclosure relate to an air disinfection systems, and in particular to microwave enhanced air disinfection systems.

### BACKGROUND

Air can include contaminants. Contaminants can include particulate matter, ground-level ozone, carbon, monoxide, sulfur dioxide, nitrogen dioxide, and lead. Other contaminants include microorganisms (e.g., living and non-living) and agents that cause infectious diseases.

### BRIEF DESCRIPTION OF THE DRAWINGS

The present disclosure is illustrated by way of example, and not by way of limitation, in the figures of the accompanying drawings in which like references indicate similar elements. It should be noted that different references to “an” or “one” embodiment in this disclosure are not necessarily to the same embodiment, and such references mean at least one.

FIG. 1 is a block diagram illustrating an exemplary system architecture, according to certain embodiments.

FIGS. 2A-B are block diagrams illustrating microwave enhanced air disinfection (MEAD) systems, according to certain embodiments.

FIGS. 3A-B are cross-sectional views of a MEAD system, according to certain embodiments.

FIGS. 4A-B are cross-sectional views of a MEAD system, according to certain embodiments.

FIG. 5 illustrates a data set generator to create data sets for a machine learning model associated with a MEAD system, according to certain embodiments.

FIG. 6 is a block diagram illustrating determining predictive data for a MEAD system, according to certain embodiments.

FIGS. 7A-E illustrate flow diagrams of methods associated with a MEAD system, according to certain embodiments.

FIG. 8 is a block diagram illustrating a computer system, according to certain embodiments.

### DETAILED DESCRIPTION OF EMBODIMENTS

Embodiments described herein are related to control of microwave enhanced air disinfection (MEAD) systems.

Safe breathable air is a basic human need. The safety of indoor air is now one of the most important issues facing governments, business operators, and consumers worldwide. Even before the severe acute respiratory syndrome coronavirus 2 (SARS-CoV-2) (e.g., coronavirus disease 2019 (COVID-19), novel coronavirus) crisis began, indoor air quality was recognized as an emerging global health issue. The World Health Organization has estimated that one in every eight people die due to factors attributable to poor indoor air. However, since most of these deaths occur in developing countries, indoor air safety has not been a focus of global attention until the COVID-19 pandemic.

Air can include many contaminants including particulate matter (e.g., particles), ground-level ozone, carbon, monoxide, sulfur dioxide, nitrogen dioxide, lead, microorganisms (e.g., living and non-living), viruses, allergens, and agents. Contaminants in the air can harm human health, harm the environment, and cause property damage.

Microorganisms (e.g., microscopic organisms) live in almost every habitat around the world. Pathogens (e.g., infectious agent, something that causes a disease, living and non-living organisms, etc.) include infectious microorganisms and agents, such as virus (e.g., non-enveloped virus, enveloped virus), bacterium, protozoan, prion, viroid, and fungus. For example, some pathogenic bacteria cause diseases such as plague, tuberculosis, and anthrax. In another example, some protozoan parasites cause diseases such as malaria, sleeping sickness, dysentery, and toxoplasmosis. In another example, some fungi cause diseases such as ring worm, candidiasis, or histoplasmosis. Some pathogenic viruses cause influenza virus (e.g., the flu), yellow fever, COVID-19, and the like.

COVID-19 and other diseases such as influenza and the common cold have been shown to be readily transmitted by airborne pathogens. Some pathogens are spread via small droplets produced by coughing, sneezing, and talking. The droplets travel through the air and some contaminate surfaces. People can become infected by coming into contact with the droplets in the air or by touching a contaminated surface and then touching their face (e.g., eyes, nose, and/or mouth). In some instances, pathogens may be spread by an infected person before and while showing symptoms.

Some pathogens (e.g., the influenza virus) spread around the world in periodical outbreaks, resulting in millions of cases of severe illness and hundreds of thousands of deaths. Some pathogens have vaccines or specific antiviral treatments, while others do not. Pandemics (e.g., COVID-19) are a spread by a pathogen causing a disease across a large region, affecting a substantial number of people within a short period of time.

Conventionally, air is periodically circulated through indoor areas (e.g., one or more rooms in a building). Conventional air circulation systems include a filter to collect some particles that are in the air that is being circulated. These conventional filters are periodically replaced. Conventional filters that do not cause much restriction on airflow collect less particles than conventional filters that cause more restriction on airflow. As filters collect more and more particles over time, the filters cause more and more restriction on air flow. Increased restriction on airflow can damage air treatment systems (e.g., cause freezing of cooling coils), decrease user comfort (e.g., provide less airflow), decrease air circulation, and the like. Conventional filters do not remove some contaminants from the air.

Conventional approaches are only partial solutions. Conventional filters capture but do not destroy contaminants (e.g., so that the contaminants no longer pose a threat) and require frequent replacement adding cost and creating a disposal hazard. Conventional filters are unable to capture small particles (e.g., smaller than 30 nanometers (nm) in size). Viruses like COVID-19 are small in size (e.g., significantly smaller than 30 nm) and are often found in droplets and particles also small in size (e.g., smaller than 30 nm in size) and can escape even the most robust conventional filtration systems. Further, as collected moisture droplets dry and break-up, fragments can escape the filter and pose a significant additional infection risk. Some conventional filtration systems are fundamentally slow, often requiring hours to clean a room-sized space after a single contamination. As a result, conventional approaches are unsuited for real-world applications. Because there is no effective means of neutralizing airborne COVID-19 available today, governments worldwide have been forced to implement policies to mitigate the spread of the disease, causing devastating economic damage and leaving businesses and consumers frantically searching for solutions. As such, there is an immediate and unmet need for air purifying products that can effectively destroy airborne contaminants like COVID-19.

The devices, systems, and methods disclosed herein provide control of a MEAD system. A processing device (e.g., controller of a MEAD system, of a server device, a client device, etc.) receives sensor data associated the MEAD system. The MEAD system includes a multi-component filter and a microwave generator that generates microwave energy. In some embodiments, the MEAD system includes a fan to provide airflow through the multi-component filter. In some embodiments, the sensor data is received from one or more sensors of the MEAD system located proximate the multi-component filter (e.g., a sensor proximate an inlet, a sensor proximate an outlet, a sensor proximate off-gassing of the contaminants from the multi-component filter, etc.). As airflow goes through the multi-component filter, contaminants from the airflow become trapped (e.g., adsorbed, collected) on the multi-component filter. At least a portion of the contaminants are destroyed at least one of directly or indirectly via the microwave energy. In some embodiments, at least a portion of the multi-component filter is heated by the microwave energy to destroy (e.g., oxidize, destroy, destroy cell structure of) contaminants from the airflow (e.g., contaminants directly destroyed via microwave energy). In some embodiments, at least a portion of the multi-component filter (e.g., zeolites, metal oxides) is activated via the multi-component filter to destroy contaminants (e.g., destroy microbes, oxidize VOCs, etc.) from the airflow (e.g., contaminants indirectly destroyed via microwave energy). In some embodiments, one or more properties of the multi-component filter (e.g., zeolites, metal oxides) may remove (e.g., destroy) contaminants (e.g., with or without airflow). In some embodiments, the microwave energy catalyzes reactions (e.g., with temperatures lower than conventional temperatures used to produce reactions, provides lower temperature of reaction, directly and/or indirectly destroys contaminants). In some embodiments, the contaminants are destroyed by one or more reactions (e.g., substantially simultaneous reactions, destroying via heating and activated portions of the multi-component filter).

In some embodiments, the sensor data is associated with the off gas of destroying the contaminants trapped on the multi-component filter. The processing device further causes, based on the sensor data, performance of a corrective action associated with the MEAD system.

In some embodiments, a machine learning model is trained based on historical sensor data and historical performance data associated with one or more MEAD systems. In some embodiments, the historical sensor data is associated with the off gas from the one or more MEAD systems responsive to removing (e.g., destroying, off gassing) contaminants trapped on corresponding multi-component filters (e.g., contaminants having been trapped on the corresponding multi-component filters by flowing airflow including the contaminants through the corresponding multi-component filters) by providing microwave energy to corresponding multi-component filters. In some embodiments, the historical performance data is associated with quality of the airflow (e.g., amount and/or type of contaminants in the airflow) entering the MEAD system, status of the multi-component filter (e.g., type and/or amount of contaminants trapped on the multi-component filter), quality of the airflow leaving the MEAD system, status of the microwave generator (e.g., how often the microwave generator is actuated to generate microwave energy, the frequency of the microwave energy, power consumption of the microwave generator, etc.). In some examples, the historical performance data includes a quantity of contaminants in the airflow entering a MEAD system, a frequency (e.g., megahertz) of the microwaves in the microwave energy, how often the microwave generator is actuated, and/or the like.

In some embodiments, the processing device provides sensor data associated with a MEAD system to the trained machine learning model and obtains, from the trained machine learning model, one or more outputs indicative of predictive data (e.g., predictive performance data). In some embodiments, the sensor data is associated with the off gassing of the MEAD system and the predictive data is associated with quality of airflow entering the MEAD system. The processing device may cause performance of the corrective action based on the predictive data.

In some embodiments, the corrective action includes one or more of causing the microwave generator of the MEAD system to generate the microwave energy (e.g., for a first quantity of time, at set intervals, etc.), causing the fan of the MEAD system to provide airflow through the MEAD system (e.g., for a quantity of time, at set intervals, at a particular flowrate, etc.), causing at least a portion of the multi-component filter to be replaced, causing interruption of generation of the microwave energy, causing an alert to be provided, and/or the like.

In some embodiments, a processing device (e.g., of a MEAD system, of a server device, of a client device, of a gateway device, etc. identifies a schedule to operate a MEAD system. The schedule may indicate when the MEAD system is to generate microwave energy at particular power settings, when the MEAD system is to provide particular airflow via the fan, etc. The processing device causes, based on the schedule, intermittent generation of microwave energy by a microwave generator of the MEAD system. The processing device receives sensor data or user input. The MEAD system determines whether the sensor data or user input matches the schedule. In some examples, the schedule indicates that there is to be less than a threshold value of sensor data from the sensor proximate the off-gassing. In some examples, the schedule indicates there is to be less than a threshold distance value of a difference in sensor data between a sensor proximate the inlet and a sensor proximate the outlet. In some examples, the schedule indicates a pattern of user input. Responsive to the sensor data or user input matching the schedule, the processing device continues using the schedule. Responsive to the sensor data or user input not matching the schedule, the processing device updates the schedule based on the sensor data or the user input. In some examples, responsive to determining the sensor data meets a threshold value or the difference in sensor data meets a threshold distance value (e.g., indicating more than a threshold amount of contaminants), the processing device updates the operation of the MEAD system (e.g., increases power to the microwave generator, increases the duration of operation of the microwave generator, increases how often the microwave generator runs, etc.). In some examples, responsive to determining the user input does not match the patterns in the schedule, the processing device causes the schedule to be updated based on the new user input (e.g., the new pattern of user input).

The systems, devices, and methods disclosed herein have advantages over conventional solutions. The microwave generator of the MEAD system is actuated intermittently based on sensor data which saves energy and wear-and-tear of the MEAD system while improving quality of airflow and protecting health of occupants. Predictive data associated with quality of airflow may be generated for performance of corrective actions which can also improve quality of airflow and protect health of occupants. The MEAD system removes more contaminants, removes smaller contaminants, and destroys contaminants compared to conventional systems that trap less contaminants, do not trap as small of contaminants, and do not destroy the contaminants. This allows the MEAD system to have greater improvement to quality of airflow and better protect health of occupants compared to conventional systems. The MEAD system destroys contaminants by heating the multi-component filter via microwave energy, by activating one or more portions (e.g., metal oxides, zeolites, etc.) of the multi-component filter via microwave energy, and so forth. The technology of the MEAD system has been shown to kill aerosolized biological agents like Escherichia coli (E. coli), Escherichia virus MS2, and Bacillus Subtilis, which are commonly used to model COVID-19 and other dangerous pathogens, in 90 seconds, which is much faster (e.g., 20-50 times faster) than conventional systems. This allows the MEAD system to provide real-time purification of indoor air. Destruction of contaminants by the MEAD system avoids frequent filter replacement of conventional systems and avoids air restriction caused by filters that need to be replaced in conventional systems. This also allows the MEAD system to have thinner filters than filters in some conventional systems, which allows the MEAD system to have less restriction on airflow. The reduced restriction on airflow of the MEAD system decreases damage to air treatment systems, increases air circulation, and increases user comfort. The MEAD system may generate microwave energy intermittently via the microwave generator which decreases energy consumption.

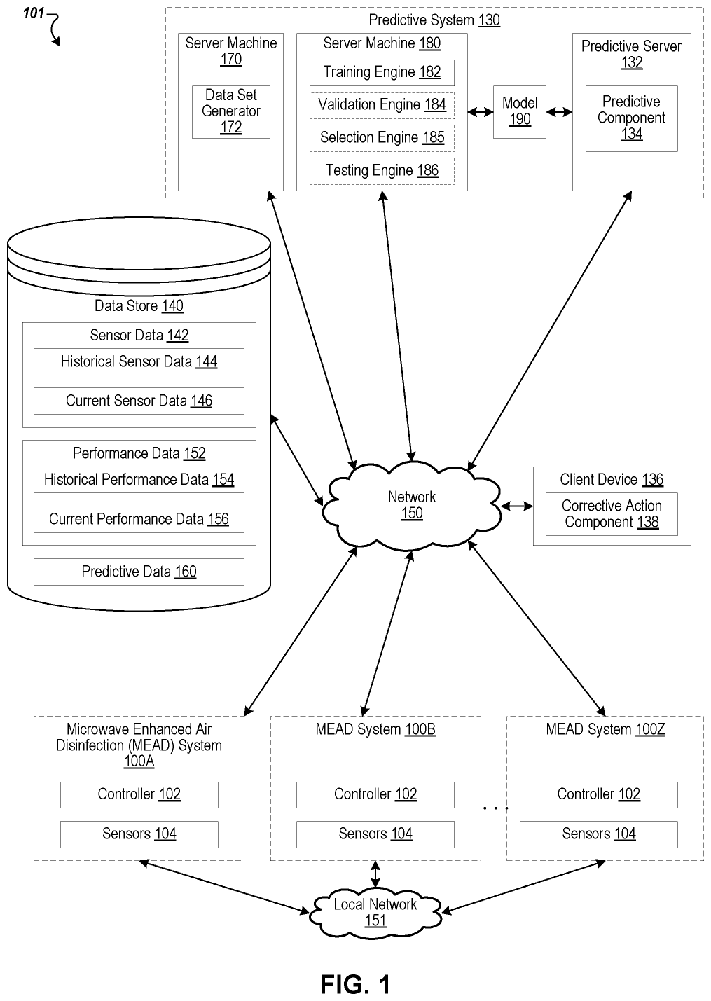

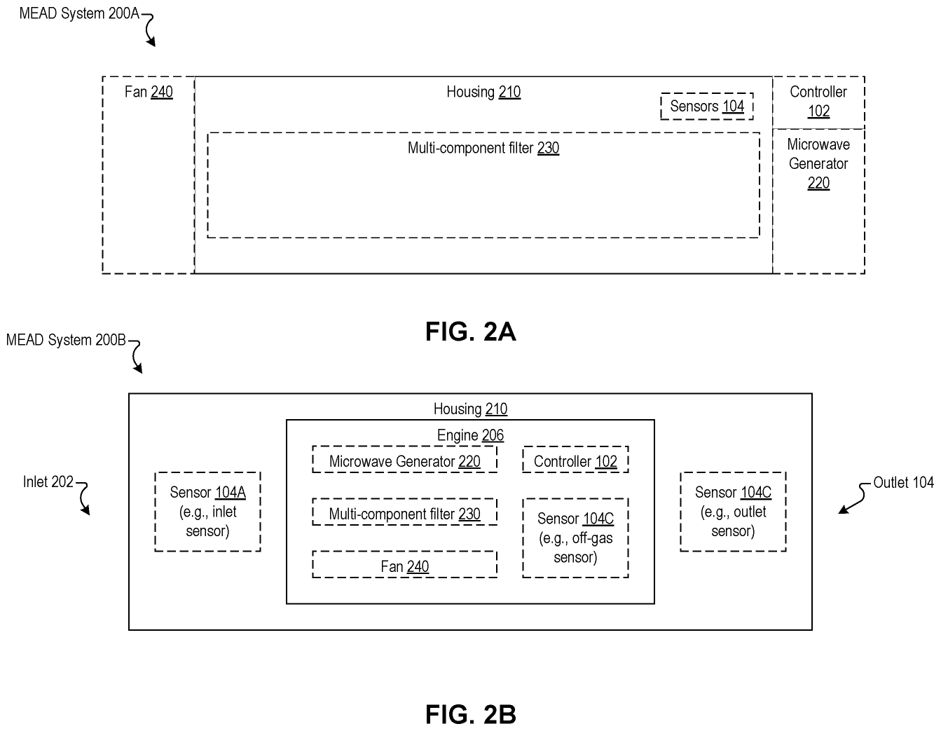

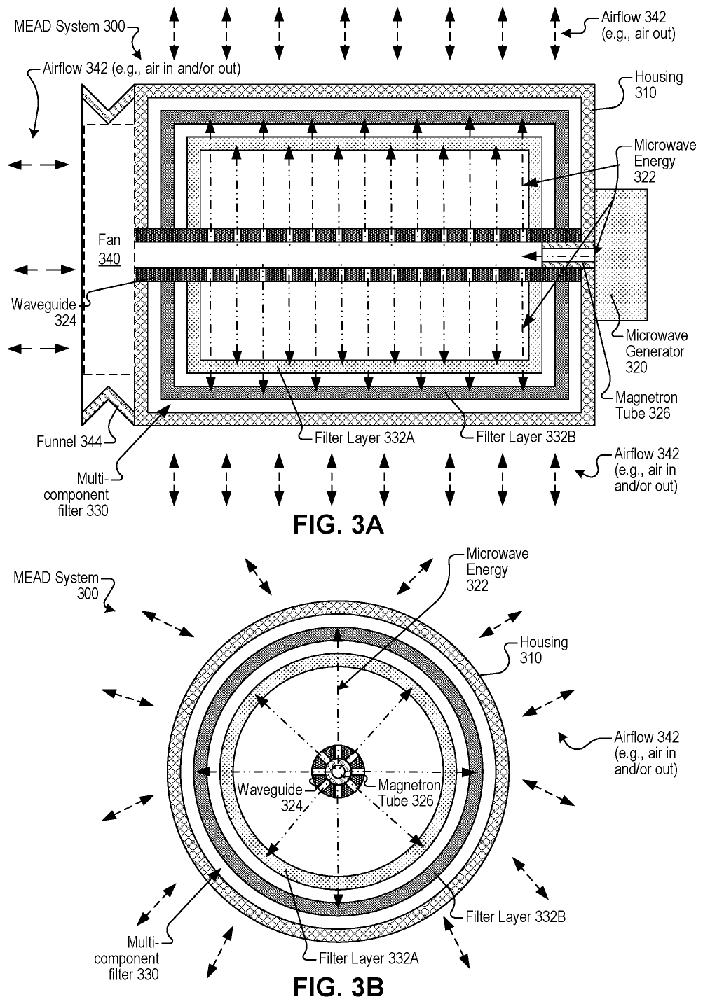

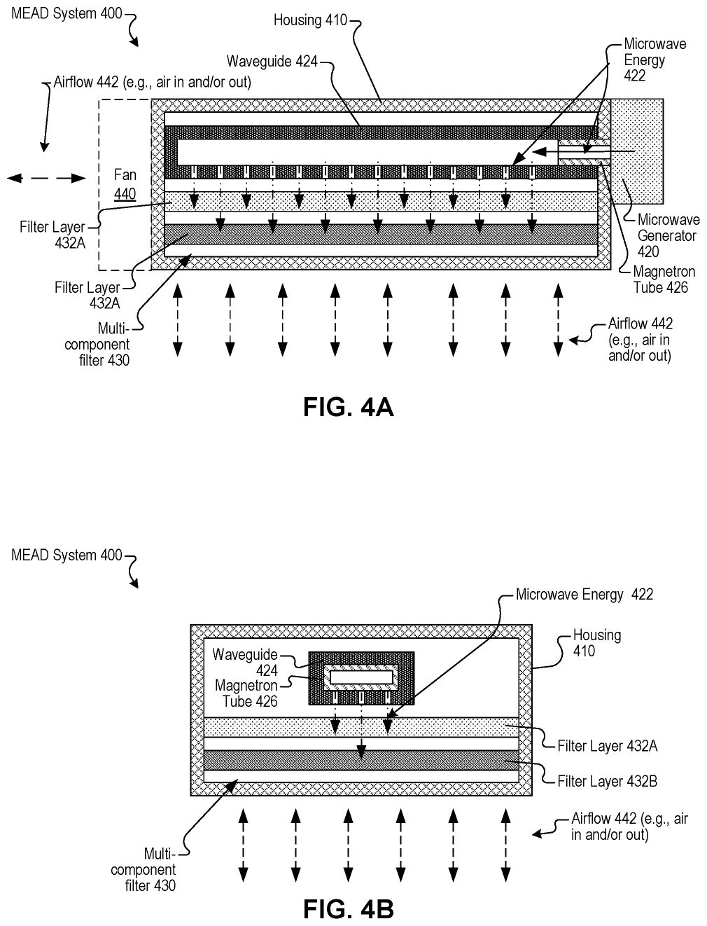

FIG. 1 is a block diagram illustrating an exemplary system 101 (exemplary system architecture), according to certain embodiments. The system 101 includes one or more MEAD systems 100 (e.g., MEAD system 200 of FIGS. 2A-B, MEAD system 300 of FIGS. 3A-B, MEAD system 400 of FIGS. 4A-B), predictive server 132, client device 136, and data store 140. In some embodiments, predictive server 132 is part of predictive system 130. In some embodiments, predictive system 130 further includes server machines 170 and 180.

In some embodiments, one or more of MEAD systems 100, client device 136, predictive server 132, data store 140, server machine 170, and/or server machine 180 are coupled to each other via a network 150 (e.g., for generating predictive data 160, for controlling MEAD systems 100, for performing corrective actions, etc.). In some embodiments, network 150 is a public network that provides client device 136 with access to the MEAD systems 100, predictive server 132, data store 140, and other publically available computing devices. In some embodiments, network 150 is a private network that provides client device 136 access to MEAD systems 100, predictive server 132, data store 140, and other privately available computing devices. In some embodiments, network 150 includes one or more Wide Area Networks (WANs), Local Area Networks (LANs), wired networks (e.g., Ethernet network), wireless networks (e.g., an 802.11 network or a Wi-Fi® network), cellular networks (e.g., a Long Term Evolution (LTE) network), radar units, transmission antenna, reception antenna, microwave transmitter, microwave receiver, sonar devices, Lidar devices, routers, hubs, switches, server computers, cloud computing networks, and/or a combination thereof.

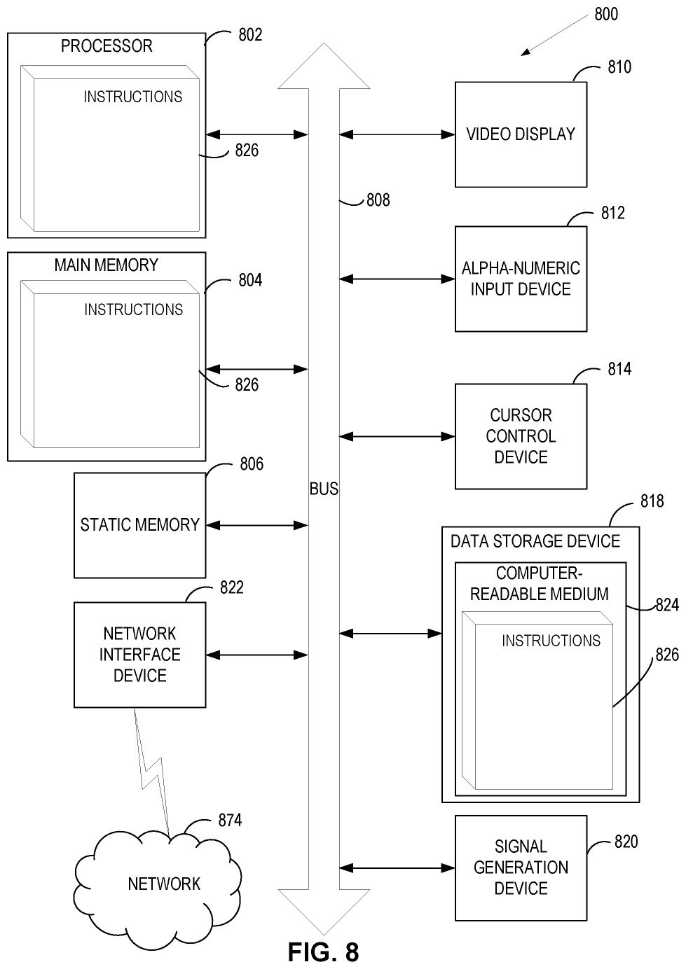

MEAD system 100 includes a multi-component filter to trap contaminants in airflow through the MEAD system 100. The MEAD system further includes a microwave generator to produce microwave energy to heat and/or activate at least a portion of the multi-component filter to remove (e.g., destroy, off gas) the contaminants from the multi-component filter. Each MEAD system includes a controller 102 (e.g., see computer system 800 of FIG. 8) and one or more sensors 104. In some embodiments, the sensors 104 provide sensor data 142 associated with the MEAD system 100 (e.g., properties of the off gassing of the MEAD system 100, temperature of the MEAD system 100, airflow through the MEAD system 100, pressure within the MEAD system 100, conditions outside of the MEAD system 100, etc.). In some embodiments, the controller 102 controls the MEAD system 100 based on the sensor data 142 from the sensors 104. In some embodiments, the controller 102 transmits the sensor data 142 to one or more of other MEAD systems 100, client device 136, data store 140, predictive system 130, etc. In some embodiments, controller 102 receives instructions (e.g., to perform a corrective action) from one or more of other MEAD systems 100, client device 136, data store 140, predictive system 130, etc. In some embodiments, controller 102 receives user input via one or more of a user interface of the MEAD system 100, via client device 136, via predictive system 130, etc. to control the MEAD system 100.

In some embodiments, one or more MEAD systems 100A-Z communicate with each other. In some embodiments, the MEAD systems 100A receives data (e.g., instructions, schedule, sensor data, etc.) from one or more of predictive system 130, client device 136, and/or data store 140 and provides the data to the one or more MEAD systems 100B-Z. In some embodiments, a MEAD system 100A receives data from one or more other MEAD systems 100B-Z and provides the data to one or more of predictive system 130, client device 136, and/or data store 140.

In some embodiments, one or more MEAD systems 100A-Z communicate over network 150. In some embodiments, one or more MEAD systems 100A-Z communicate over a local network 151. Local network 151 may be a computing network that provides one or more communication channels between MEAD systems 100. In some examples, local network 151 is a peer-to-peer network that does not rely on a pre-existing network infrastructure (e.g., access points, switches, routers) and MEAD systems 100 replace the networking infrastructure to route communications between the MEAD systems 100. Local network 151 may be a wireless network that is self-configuring and enables MEAD systems 100 to contribute to local network 151 and dynamically connect and disconnect from local network 151 (e.g., ad hoc wireless network). In some examples, local network 151 is a computing network that includes networking infrastructure that enables MEAD systems 100 to communicate with other MEAD systems 100. The local network 151 may or may not have access to the public network (e.g., internet, network 150). For example, an access point or device that may function as an access point to enable MEAD systems 100 to communicate with one another without providing internet access. In some embodiments, the local network 151 provides access to a larger network such as network 150 (e.g., Internet). In some embodiments, local network 151 is based on any wireless or wired communication technology and may connect a first MEAD system 100 directly or indirectly (e.g., involving an intermediate device, such as an intermediate MEAD system 100) to a second MEAD system 100. The wireless communication technology may include Bluetooth®, Wi-Fi®, infrared, ultrasonic, or other technology. The wired communication may include universal serial bus (USB), Ethernet, RS 232, or other wired connection. The local network 151 may be an individual connection between two MEAD systems 100 or may include multiple connections.

In some embodiments, the client device 136 includes a computing device such as Personal Computers (PCs), laptops, mobile phones, smart phones, tablet computers, netbook computers, gateway device, etc. In some embodiments, the client device 136 includes a corrective action component 138. Client device 136 includes an operating system that allows users to one or more of generate, view, or edit data (e.g., selection of a MEAD system 100, corrective actions associated with MEAD systems 100, etc.).

In some embodiments, corrective action component 138 receives user input (e.g., via a Graphical User Interface (GUI) displayed via the client device 136) of an indication associated with a MEAD system 100. In some embodiments, the corrective action component 138 transmits the indication to the predictive system 130, receives output (e.g., predictive data 160) from the predictive system 130, determines a corrective action associated with the MEAD system 100 based on the output, and causes the corrective action to be implemented. In some embodiments, the corrective action component 138 obtains sensor data 142 (e.g., current sensor data 146) associated with the MEAD system 100 (e.g., from data store 140, etc.) and provides the sensor data 142 (e.g., current sensor data 146) associated with the MEAD system 100 to the predictive system 130. In some embodiments, the corrective action component 138 stores sensor data 142 in the data store 140 and the predictive server 132 retrieves the sensor data 142 from the data store 140. In some embodiments, the predictive server 132 stores output (e.g., predictive data 160) of the trained machine learning model 190 in the data store 140 and the client device 136 retrieves the output from the data store 140. In some embodiments, the corrective action component 138 receives an indication of a corrective action from the predictive system 130 and causes the corrective action to be implemented.

In some embodiments, a corrective action is associated with one or more of Computational Process Control (CPC), Statistical Process Control (SPC) (e.g., SPC to compare to a graph of 3-sigma, etc.), Advanced Process Control (APC), model-based process control, preventative operative maintenance, design optimization, updating of operating parameters, feedback control, machine learning modification, or the like.

In some embodiments, the corrective action includes providing an alert (e.g., an alarm to replace or repair a component of the MEAD system 100 if the predictive data 160 indicates a predicted abnormality, such as an abnormality of the airflow, off gas, a component, MEAD system 100, or the like). In some embodiments, the corrective action includes providing feedback control (e.g., modifying operations responsive to the predictive data 160 indicating a predicted abnormality). In some embodiments, the corrective action includes providing machine learning (e.g., causing repair or replacement of a component of the MEAD system 100 based on the predictive data 160). In some embodiments, performance of the corrective action includes causing updates to one or more operating parameters of one or more components of the MEAD system 100. In some embodiments, the corrective action includes causing preventative maintenance.

In some embodiments, the predictive server 132, server machine 170, and server machine 180 each include one or more computing devices such as a rackmount server, a router computer, a server computer, a personal computer, a mainframe computer, a laptop computer, a tablet computer, a desktop computer, Graphics Processing Unit (GPU), accelerator Application-Specific Integrated Circuit (ASIC) (e.g., Tensor Processing Unit (TPU)), etc.

The predictive server 132 includes a predictive component 134. In some embodiments, the predictive component 134 receives sensor data 142 (e.g., receive from the client device 136, retrieve from the data store 140) and generates output (e.g., predictive data 160) for performing corrective action associated with the MEAD system 100 based on the sensor data 142. In some embodiments, the predictive component 134 uses one or more trained machine learning models 190 to determine the output for performing the corrective action based on the sensor data 142. In some embodiments, trained machine learning model 190 is trained using historical sensor data 144 and historical performance data 154.

In some embodiments, the predictive system 130 (e.g., predictive server 132, predictive component 134) generates predictive data 160 using supervised machine learning (e.g., supervised data set, labeled data, etc.). In some embodiments, the predictive system 130 generates predictive data 160 using semi-supervised learning (e.g., semi-supervised data set, a predictive percentage, etc.). In some embodiments, the predictive system 130 generates predictive data 160 using unsupervised machine learning (e.g., unsupervised data set, clustering, etc.).

In some embodiments, the sensors 104 provide sensor data 142 (e.g., historical sensor data 144, current sensor data 146) associated with MEAD system 100. In some embodiments, the sensors 104 include one or more of a pressure sensor, a flow sensor, a temperature sensor, a humidity sensor, a barometer, a light-sensing sensor, an imaging device, electrical current sensor, voltage sensor, a location sensor (e.g., global positioning system (GPS) device), and/or the like. In some embodiments, one or more sensors 104 includes sensor probes that measure sensor data 142 including chemical and physical air quality parameters. In some embodiments, the sensors 104 provide the sensor data 142 during operation of the MEAD system 100 (e.g., operation of a fan, operation of a microwave generator). In some embodiments, the sensor data 142 is used for equipment health, air treatment, energy usage, and/or the like. The sensor data 142 is received over a period of time.

In some embodiments, sensor data 142 is associated with or indicative of operating parameters such as hardware parameters (e.g., settings or components (e.g., size, type, etc.) of the MEAD system 100) or process parameters of the MEAD system 100. In some embodiments, sensor data 142 is provided while the MEAD system 100 performs operations (e.g., fan operation, microwave energy generation, etc.), before the MEAD system 100 performs operations, and/or after the MEAD system 100 performs operations. In some examples, the sensor data 142 is provided after commissioning, installation, preventative maintenance, and/or replacement of at least a portion of the MEAD system 100.

In some embodiments, the sensor data 142 (e.g., historical sensor data 144, current sensor data 146, etc.) is processed (e.g., by the client device 136 and/or by the predictive server 132). In some embodiments, processing of the sensor data 142 includes generating features. In some embodiments, the features are a pattern in the sensor data 142 (e.g., slope, width, height, peak, etc.) or a combination of sensor values from the sensor data 142 (e.g., power derived from voltage and current, etc.). In some embodiments, the sensor data 142 includes features and the features are used by the predictive component 134 for obtaining predictive data 160 for performance of a corrective action.

In some embodiments, the data store 140 is memory (e.g., random access memory), a drive (e.g., a hard drive, a flash drive), a database system, or another type of component or device capable of storing data. In some embodiments, data store 140 includes multiple storage components (e.g., multiple drives or multiple databases) that span multiple computing devices (e.g., multiple server computers). In some embodiments, the data store 140 stores one or more of sensor data 142, performance data 152, and/or predictive data 160.

Sensor data 142 includes historical sensor data 144 and current sensor data 146. In some embodiments, the sensor data 142 includes pressure data, flow data, temperature data, humidity data, barometer data, light-sensing data, image data, electrical current data, voltage data, air quality data, environmental conditions data (e.g., temperature, pressure, light, etc.), off-gas data, and/or the like. In some embodiments, the corrective action is associated with a difference between the sensor data 142 and threshold data.

Performance data 152 includes historical performance data 154 and current performance data 156. In some embodiments, the performance data 152 is data (e.g., sensor data 142) associated with the MEAD system 100 after performance of a corrective action. In some examples, the performance data 152 is pressure data, flowrate data, temperature data, off-gas data, air quality data, and/or the like (e.g., after performance of a corrective action). In some embodiments, performance data 152 includes data associated with the corrective action performed (e.g., iterations of microwave energy generation, length of time of microwave energy generation, frequency and/or power of microwave energy generated, type of repair or replacement of a component, historical corrective actions, current corrective actions, etc.). In some embodiments, the performance data 152 is a quantity of contaminants that were destroyed (e.g., based on a difference between inlet sensor data and outlet sensor data).

In some examples, the performance data 152 indicates an abnormality associated with the MEAD system 100 (e.g., quality of air entering the MEAD system, quality of air treated by the MEAD system, off gassing from the MEAD system, component failure, maintenance, energy usage, variance of a component compared to similar components, etc.). In some embodiments, the performance data 152 is associated with yield (e.g., yield of treated airflow, yield of off gassing, yield of contaminant removal, etc.), average yield, predicted yield, predicted abnormality of product, and/or the like. In some examples, responsive to yield over a first period of time being a first amount, the client device 136 causes a corrective action based on a prediction that product over an upcoming period of time is to have the same yield.

Historical data includes one or more of historical sensor data 144 and/or historical performance data 154 (e.g., at least a portion for training the machine learning model 190). Current data includes one or more of current sensor data 146 and/or current performance data 156 (e.g., at least a portion to be input into the trained machine learning model 190 subsequent to training the model 190 using the historical data) for which predictive data 160 is generated (e.g., for performing corrective actions). In some embodiments, the current data is used for retaining the trained machine learning model 190.

In some embodiments, predictive data 160 is associated with predictive performance data of the MEAD system 100 (e.g., predicted quality of air to be treated, predicted contaminants in the air to be treated, predictive quality of treated air, amount of treated air, pressure levels, flow rates, energy consumption, and/or the like). In some embodiments, the predictive data 160 is predictive performance data of the MEAD system after performing a particular corrective action.

Performing operations that result in poor quality of product (e.g., poor quality of treated air) is costly in time, energy, components, the MEAD system 100, etc. By inputting sensor data 142, receiving output of predictive data 160, and performing a corrective action based on the predictive data 160, system 101 has the technical advantage of avoiding producing poor air quality.

Performing operations that result in failure of the components of the MEAD system 100 is costly in downtime, damage to equipment, express ordering replacement components, etc. By inputting sensor data 142, receiving output of predictive data 160, and performing corrective action (e.g., replacement, repair, preventative maintenance, etc. of components) based on the predictive data 160, system 101 has the technical advantage of avoiding the cost of one or more of unexpected component failure, unscheduled downtime, productivity loss, unexpected equipment failure, and the like.

In some embodiments, operating parameters are suboptimal (e.g., too few of iterations of generating microwave energy and/or actuating fan, etc.) which has costly results of increased resource (e.g., energy, etc.) consumption, increased amount of time to output the air, increased component failure, etc. By inputting the sensor data 142 into the trained machine learning model 190, receiving an output of predictive data 160, and performing (e.g., based on the predictive data 160) a corrective action of updating operating parameters (e.g., iterations, schedule, etc. for microwave energy generation and/or fan operation), system 101 has the technical advantage of using optimal operating parameters to avoid costly results of suboptimal operating parameters.

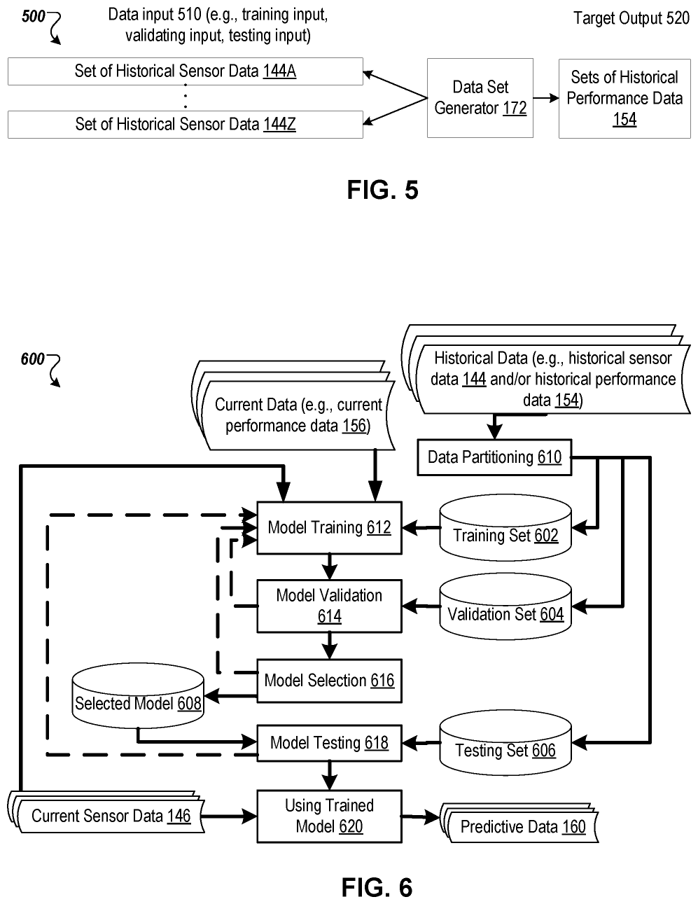

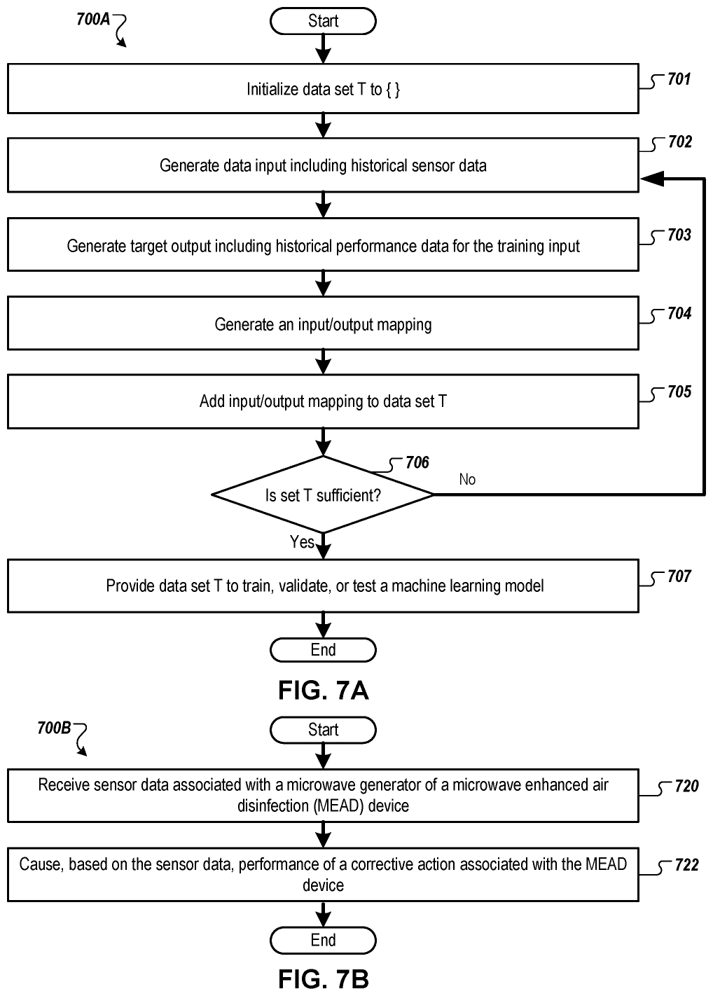

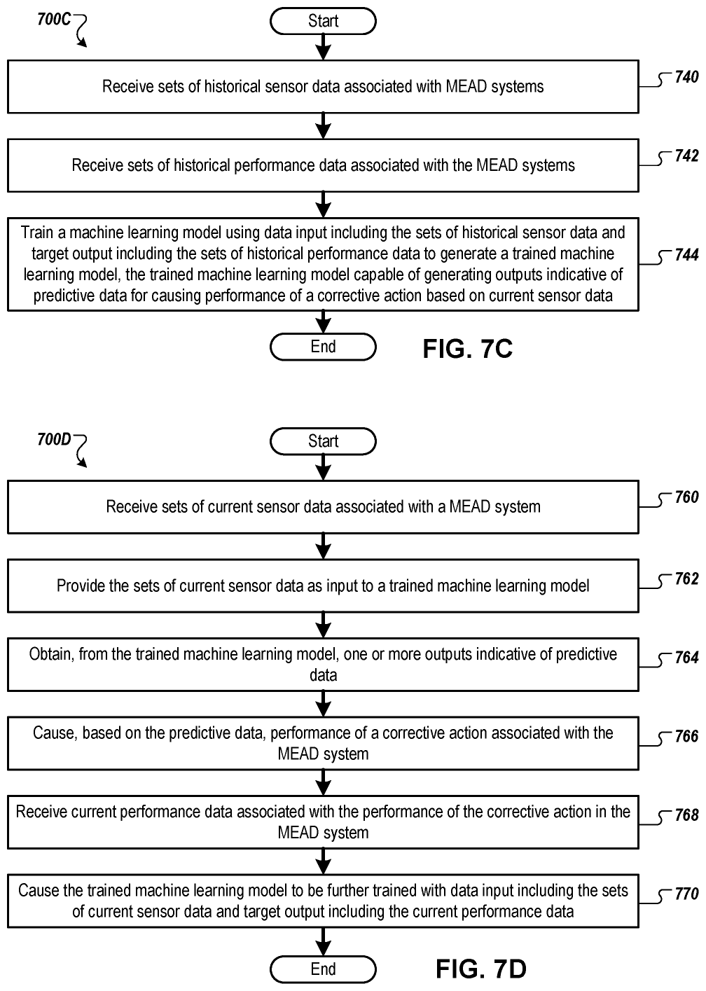

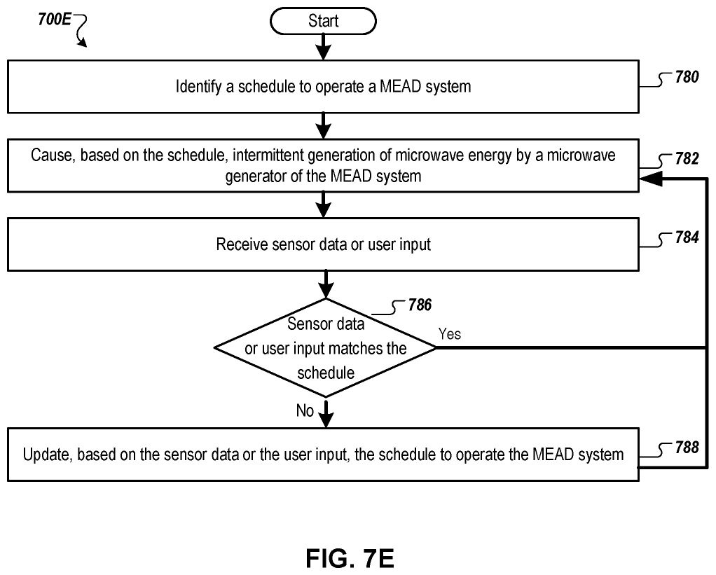

In some embodiments, predictive system 130 further includes server machine 170 and server machine 180. Server machine 170 includes a data set generator 172 that is capable of generating data sets (e.g., a set of data inputs and a set of target outputs) to train, validate, and/or test a machine learning model(s) 190. Some operations of data set generator 172 are described in detail below with respect to FIGS. 5 and 7A. In some embodiments, the data set generator 172 partitions the historical data (e.g., historical sensor data 144 and historical performance data 154) into a training set (e.g., sixty percent of the historical data), a validating set (e.g., twenty percent of the historical data), and a testing set (e.g., twenty percent of the historical data). In some embodiments, the predictive system 130 (e.g., via predictive component 134) generates multiple sets of features. In some examples, a first set of features corresponds to a first set of types of sensor data (e.g., from a first set of sensors, first combination of values from first set of sensors, first patterns in the values from the first set of sensors) that correspond to each of the data sets (e.g., training set, validation set, and testing set) and a second set of features correspond to a second set of types of sensor data (e.g., from a second set of sensors different from the first set of sensors, second combination of values different from the first combination, second patterns different from the first patterns) that correspond to each of the data sets.

Server machine 180 includes a training engine 182, a validation engine 184, selection engine 185, and/or a testing engine 186. In some embodiments, an engine (e.g., training engine 182, a validation engine 184, selection engine 185, and a testing engine 186) refers to hardware (e.g., circuitry, dedicated logic, programmable logic, microcode, processing device, etc.), software (such as instructions run on a processing device, a general purpose computer system, or a dedicated machine), firmware, microcode, or a combination thereof. The training engine 182 is capable of training a machine learning model 190 using one or more sets of features associated with the training set from data set generator 172. In some embodiments, the training engine 182 generates multiple trained machine learning models 190, where each trained machine learning model 190 corresponds to a distinct set of features of the training set (e.g., sensor data from a distinct set of sensors). In some examples, a first trained machine learning model was trained using all features (e.g., X1-X5), a second trained machine learning model was trained using a first subset of the features (e.g., X1, X2, X4), and a third trained machine learning model was trained using a second subset of the features (e.g., X1, X3, X4, and X5) that partially overlaps the first subset of features.

The validation engine 184 is capable of validating a trained machine learning model 190 using a corresponding set of features of the validation set from data set generator 172. For example, a first trained machine learning model 190 that was trained using a first set of features of the training set is validated using the first set of features of the validation set. The validation engine 184 determines an accuracy of each of the trained machine learning models 190 based on the corresponding sets of features of the validation set. The validation engine 184 discards trained machine learning models 190 that have an accuracy that does not meet a threshold accuracy. In some embodiments, the selection engine 185 is capable of selecting one or more trained machine learning models 190 that have an accuracy that meets a threshold accuracy. In some embodiments, the selection engine 185 is capable of selecting the trained machine learning model 190 that has the highest accuracy of the trained machine learning models 190.

The testing engine 186 is capable of testing a trained machine learning model 190 using a corresponding set of features of a testing set from data set generator 172. For example, a first trained machine learning model 190 that was trained using a first set of features of the training set is tested using the first set of features of the testing set. The testing engine 186 determines a trained machine learning model 190 that has the highest accuracy of all of the trained machine learning models based on the testing sets.

In some embodiments, the machine learning model 190 refers to the model artifact that is created by the training engine 182 using a training set that includes data inputs and corresponding target outputs (correct answers for respective training inputs). Patterns in the data sets can be found that map the data input to the target output (the correct answer), and the machine learning model 190 is provided mappings that captures these patterns. In some embodiments, the machine learning model 190 uses one or more of Support Vector Machine (SVM), Radial Basis Function (RBF), clustering, supervised machine learning, semi-supervised machine learning, unsupervised machine learning, k-Nearest Neighbor algorithm (k-NN), linear regression, random forest, neural network (e.g., artificial neural network), etc. In some embodiments, the machine learning model 190 is a multi-variable analysis (MVA) model.

Predictive component 134 provides current sensor data 146 to the trained machine learning model 190 and runs the trained machine learning model 190 on the input to obtain one or more outputs. The predictive component 134 is capable of determining (e.g., extracting) predictive data 160 from the output of the trained machine learning model 190 and determines (e.g., extracts) confidence data from the output that indicates a level of confidence that the predictive data 160 corresponds to current performance data 156 (e.g., model 190) of the MEAD system 100 at the current sensor data 146. In some embodiments, the predictive component 134 or corrective action component 138 use the confidence data to decide whether to cause a corrective action associated with the MEAD system 100 based on the predictive data 160.

The confidence data includes or indicates a level of confidence that the predictive data 160 corresponds to current performance data 156 (e.g., model 190) of the MEAD system 100 at the current sensor data 146. In one example, the level of confidence is a real number between 0 and 1 inclusive, where 0 indicates no confidence that the predictive data 160 corresponds to current performance data 156 associated with the current sensor data 146 and 1 indicates absolute confidence that the predictive data 160 corresponds to current performance data 156 associated with the current sensor data 146. Responsive to the confidence data indicating a level of confidence below a threshold level for a predetermined number of instances (e.g., percentage of instances, frequency of instances, total number of instances, etc.) the predictive component 134 causes the trained machine learning model 190 to be re-trained (e.g., based on the current sensor data 146 and current performance data 156, etc.).

For purpose of illustration, rather than limitation, aspects of the disclosure describe the training of one or more machine learning models 190 using historical data (e.g., historical sensor data 144 and historical performance data 154) and inputting current data (e.g., current sensor data 146) into the one or more trained machine learning models 190 to determine predictive data 160 (e.g., predicting current performance data 156). In other implementations, a heuristic model or rule-based model is used to determine predictive data 160 (e.g., without using a trained machine learning model). Predictive component 134 monitors historical sensor data 144 and historical performance data 154. In some embodiments, any of the information described with respect to data inputs 510 of FIG. 5 are monitored or otherwise used in the heuristic or rule-based model.

In some embodiments, the functions of client device 136, predictive server 132, server machine 170, and server machine 180 are be provided by a fewer number of machines. For example, in some embodiments, server machines 170 and 180 are integrated into a single machine, while in some other embodiments, server machine 170, server machine 180, and predictive server 132 are integrated into a single machine. In some embodiments, client device 136 and predictive server 132 are integrated into a single machine.

In general, functions described in one embodiment as being performed by client device 136, predictive server 132, server machine 170, and server machine 180 can also be performed on predictive server 132 in other embodiments, if appropriate. In addition, the functionality attributed to a particular component can be performed by different or multiple components operating together. For example, in some embodiments, the predictive server 132 determines the corrective action based on the predictive data 160. In another example, client device 136 determines the predictive data 160 based on output from the trained machine learning model.

In some embodiments, the corrective action component 138 is part of the predictive system 130 (e.g., predictive server 132). In some embodiments, the predictive component 134 is part of the client device 136. In some embodiments, the corrective action component 138 and/or the predictive component 134 is part of the controller 102 of a MEAD system 100.

In addition, the functions of a particular component can be performed by different or multiple components operating together. In some embodiments, one or more of the predictive server 132, server machine 170, or server machine 180 are accessed as a service provided to other systems or devices through appropriate application programming interfaces (API).

In some embodiments, a “user” is represented as a single individual. However, other embodiments of the disclosure encompass a “user” being an entity controlled by a plurality of users and/or an automated source. In some examples, a set of individual users federated as a group of administrators is considered a “user.”

Although embodiments of the disclosure are discussed in terms of generating predictive data 160 to perform a corrective action associated with the MEAD system 100, in some embodiments, the disclosure can also be generally applied to verifying correct operation of components and production of product. Embodiments can be generally applied to verifying correct operation and production based on different types of data.

FIGS. 2A-B are block diagrams illustrating MEAD systems 200A-B (hereinafter MEAD system 100) (e.g., a MEAD system 100 of FIG. 1, MEAD device), according to certain embodiments. Components of FIGS. 2A and/or 2B that have similar reference numbers as components of FIG. 1 may have at least some of the same structure and/or functionality.

The MEAD system 200 includes a housing 210. In some embodiments, the MEAD system 200 is a device and the housing 210 is the device housing, where components of the MEAD system 200 are included in the housing 210 and/or are attached to the housing 210. In some embodiments, the MEAD system 200 has one or more components that are coupled (e.g., electrically coupled, fluidly coupled, etc.) to each other without being attached to the housing 210 and/or disposed in the housing 210. In some embodiments, the MEAD system 200 is a stand-alone device. In some embodiments, the MEAD system 200 is installed in conjunction with another system (e.g., in ducting of a heating ventilation and air conditioning (HVAC) system, integrated into an HVAC system, retrofit to an HVAC system, etc.).

The MEAD system 200 includes a microwave generator 220 (e.g., microwave generator with magnetron tube, solid state microwave generator, solid state digital power supply, etc.) that is coupled to the housing 210. In some embodiments, the microwave generator 220 is disposed in the housing 210. In some embodiments, the microwave generator 220 is attached to the housing 210. The microwave generator 220 generates microwave energy that is transmitted into the housing 210. In some embodiments, the MEAD system 200 includes a microwave reflective enclosure (e.g., the housing 210 is a microwave reflective enclosure, a microwave reflective enclosure is disposed in the housing, etc.). The microwave reflective enclosure prevents microwave energy from exiting the MEAD system 200. In some embodiments, the microwave generator 220 generates microwave energy intermittently (e.g., based on a schedule, on/off timer, duty cycle, based on sensor data, based on instructions, intermittent microwave energy operation, etc.). In some embodiments, the microwave generator 220 generates microwave energy continuously (e.g., continuous operation).

The MEAD system 200 includes a multi-component filter 230 that is disposed in the housing 210 (or at least partially disposed in the housing 210). Airflow passes through the multi-component filter 230 and contaminants from the airflow are trapped by the multi-component filter 230. At least a portion of the multi-component filter 230 is configured to be heated and/or activated by the microwave energy generated by the microwave generator 220 to remove (e.g., oxidize, destroy, off-gas, etc.) contaminants from the airflow (e.g., contaminants trapped in the multi-component filter 230). The contaminants are heated, destroyed, and/or off-gassed.

In some embodiments, the multi-component filter 230 is made of two or more filter materials, where each of the filter materials has a different function. In some embodiments, the multi-component filter 230 has two or more layers, where each of the layers is made of a different filter material. In some embodiments, the multi-component filter 230 uses one or more heterogeneous structures instead of or in addition to discrete filter layers. In some embodiments, the multi-component filter 230 is a heterogeneous mix (e.g., heterogeneous structure) of two or more filter materials that each have a different function. In some embodiments, the multi-component filter 230 includes one or more of a pre-filter, a microwave-absorbing material, a metal oxide (e.g., copper oxide, zinc oxide, titanium oxide, etc.), a metal carbide (e.g., silicon carbide, etc.), zeolites, a molecular sieve, a material without organic binders, a material with inorganic binders, a HEPA filter, and/or the like. In some embodiments, a layer of metal oxide is located closest to the microwave energy (e.g., is heated and/or activated the most), a HEPA filter layer is located furthest from the microwave energy (e.g., heated and/or activated the least, not heated and/or activated), and a zeolite layer is located between the metal oxide layer and the HEPA filter layer. In some embodiments, the metal layer is used to remove and destroy living and non-living microorganisms, the molecular sieve (e.g., zeolite layer) is used to remove VOCs, and the HEPA filter layer is used to remove remaining contaminants.

In some embodiments, the multi-component filter 230 is less than about 4 inches deep (e.g., less than 4 inches from where airflow enters the multi-component filter to where the airflow leaves the multi-component filter to exit the MEAD system 200). In some embodiments, the multi-component filter is less than about 3 inches deep. In some embodiments, the multi-component filter is less than about 2 inches deep. In some embodiments, the multi-component filter is about 2 to 4 inches deep. In some embodiments, the multi-component filter is 12 to 16 inches in length (e.g., the waveguide is 12 to 16 inches in length).

In some embodiments, the MEAD system 200 has a fan 240 coupled to the housing 210. In some embodiments, the MEAD system 200 has a fan 240 disposed within the housing 210. In some embodiments, the fan 240 provides the airflow into the housing 210 to be filtered by the multi-component filter 230 and the same fan 240 provides the airflow to cool the microwave generator 220. In some embodiments, the MEAD system 200 does not have a fan (e.g., airflow is provided by a component outside of the MEAD system 200, such as by a blower of an HVAC system). In some embodiments, the fan 240 (e.g., a suction fan) pulls the airflow into the housing 220 and causes the airflow to exit the housing 220 through the fan 240 (e.g., airflow goes through multi-component filter 230 before going through fan 240). In some embodiments, the fan 240 pushes the airflow into the MEAD system 200 and causes the airflow to exit the MEAD system 200 through the housing 220 (e.g., airflow goes through fan 240 before going through multi-component filter 230). In some embodiments, the fan 240 is configured to switch operation between pushing airflow and pulling airflow (e.g., to loosen contaminants in the multi-component filter 230). In some embodiments, the MEAD system 200 includes a pressure sensor to measure pressure drop across the multi-component filter 230. Responsive to the controller 102 determining, based on pressure data from the pressure sensor, that the pressure drop meets a threshold pressure drop, the controller 102 may cause one or more corrective actions (e.g., cause the fan 240 to increase airflow, cause the fan 240 to alternate airflow between pushing and pulling, provide an alert to clean or replace a portion of the MEAD system 200, etc.).

In some embodiments, the fan 240 is a quiet fan to pull air through the MEAD system 200. In some embodiments, the multi-component filter 230 includes a HEPA filter that removes about 99.97% of all small particles before discharge. In some embodiments, the multi-component filter 230 includes a filter matrix that effectively collects aerosols, odors, and other violates. In some embodiments, the filter is combined with materials (e.g., via inorganic binders) that react to microwave energy and are activated (e.g., heat to temperatures high enough) to destroy contaminants, such as viruses and VOCs. The microwave generator 220 (e.g., with a waveguide and/or magnetron tube) is used to distribute microwave energy evenly across filter materials of the multi-component filter 230. In some embodiments, contaminants (e.g., virus aerosols and VOCs) are collected on the multi-component filter 230 (e.g., filtration media) that can be heated and/or activated by microwave energy (e.g., microwaves) on a periodic cycle so that the microwave system is not operating continuously. In some embodiments, the MEAD system 200 operates an alternating adsorption-microwave regeneration cycle (e.g., multi-component filter 230 adsorbs contaminants and then the microwave generator 220 generates microwave energy to destroy the contaminants on the multi-component filter 230 to regenerate the multi-component filter 230).

In some embodiments, the MEAD system 200 includes a controller 102 disposed in the housing 210 or coupled to the housing 210. In some embodiments, the microwave generator 220 includes a controller 102. The controller 102 includes one or more of a processing device, memory, sensors, wireless component, a user interface, and/or the like. In some embodiments, the controller 102 includes one or more of the components of computer system 600 of FIG. 6. In some embodiments, the controller actuates (e.g., turns on, turns off, adjusts fan speed, adjusts microwave energy generation, etc.) the microwave generator 220 and/or fan 240 based on one or more of a schedule, user input, sensor data, etc.

In some embodiments, the MEAD system 200 includes one or more sensors 104 coupled to or within the housing 210. In some embodiments, the one or more sensors 104 are disposed in the airflow after one or more portions of the multi-component filter 230 (e.g., after the airflow has been at least partially filtered). As the contaminants are trapped in the multi-component filter 230 and destroyed by the microwave energy heating and/or activating the multi-component filter 230, the contaminants are off-gassed. In some embodiments, the one or more sensors 104 are located to provide sensor data associated with the off-gassed contaminants.

In some embodiments, the fan 240 is disposed at a first distal end of the housing 210 and the microwave generator 220 is disposed at a second distal end of the housing 210 (e.g., see FIG. 2A). The fan 240 may pull airflow into the MEAD system via the housing 210 (e.g., the airflow exits through the fan 240) and/or the fan 240 may provide airflow into the MEAD system through the fan 240 (e.g., the airflow exits through the housing 210).

In some embodiments, the MEAD system 200 includes an inlet 202 (e.g., large airflow inlet) and an outlet 204 (e.g., large airflow outlet) that are substantially in line with each other (e.g., the inlet and the outlet are disposed along a common axis, a central axis substantially runs through a center of the inlet and a center of the outlet, see FIG. 2B, etc.). One or more components (e.g., an engine 206) may be disposed between the inlet 202 and the outlet 204 (e.g., between the inlet and outlet that are in line with each other). The engine 206 may include one or more of the microwave generator 220, multi-component filter 230, fan 240, controller 102, one or more sensors 104, etc.

In some embodiments, the sensors 104 include a sensor 104A is disposed proximate an inlet (e.g., inlet 202, housing 210) of the MEAD system 200, a sensor 104B is disposed proximate the off-gassing (e.g., multi-component filter 230, a portion of the multi-component filter 130 that reaches a higher temperature than other portions of the multi-component filter 130 to trigger combustion, etc.), and a sensor 104C located proximate the outlet (e.g., outlet 204, fan 240) of the MEAD system 200. The controller 102 may receive sensor data from the sensors 104 and cause a corrective action based on the sensor data or differences between the sensor data from different sensors 104. In some examples, responsive to determining, based on sensor data (e.g., off-gassing sensor data) from sensor 104B, that a threshold amount of contaminants or a certain type of contaminants are in the airflow, the controller 102 may cause the MEAD system 200 to continue operating (e.g., generating microwave energy and airflow, increase power provided to the microwave generator 220, increase airflow, etc.). Responsive to determining, based on sensor data from sensor 104B, that a threshold amount of contaminants or certain types of contaminants are not in the airflow, the controller 102 may cause the MEAD system 200 to stop or slow down operation (e.g., decrease power to microwave generator 220, decrease airflow via fan 240, stop generation of microwave energy and/or airflow, etc.).

In some examples, responsive to determining, based on sensor data (e.g., inlet sensor data) from sensor 104A and sensor data (e.g., outlet sensor data 104C) from sensor 104C, a difference value that exceeds a threshold difference value, the controller may cause the MEAD system 200 to continue operating (e.g., generating microwave energy and airflow). Responsive to determining, based on sensor data from sensors 104A and 104C, that a threshold difference value is not met, the controller 102 may cause the MEAD system 200 to stop or slow down operation (e.g., decrease power to microwave generator 220, decrease airflow via fan 240, stop generation of microwave energy and/or airflow, etc.).

In some embodiments, the controller 102 may cause the fan 240 to reverse airflow (e.g., inlet 202 is used as an outlet and outlet 204 is used as an inlet). Responsive to reversing airflow, the controller 102 may use sensor data from sensor 104C as inlet sensor data and may use sensor data from sensor 104A as outlet sensor data.

In some embodiments, the controller 102 may cause the MEAD system 200 to operate continuously (e.g., generate microwave energy via microwave generator 220 and generate airflow via fan 240 responsive to being turned on). In some embodiments, the controller 102 may cause the MEAD system 200 to operate intermittently (e.g., based on a timer, based on a schedule, based on sensor data, etc.).

In some embodiments, one or more MEAD systems 200 communicate, via a network, with a processing device (e.g., a server device, another MEAD system 200, client device, gateway device, etc.) that is remote from the one or more MEAD systems 200. The processing device may receive sensor data from the one or more MEAD systems 200 and provide instructions to (e.g., control, direct operation of) one or more MEAD systems 200. In some examples, responsive to receiving sensor data indicative of a certain contaminant (e.g., influenza, etc.), the processing device may cause multiple MEAD systems 200 (e.g., in a region, in a space, in a building) to perform an operation (e.g., increased power to the microwave generator 220, increased airflow, more frequent operation, etc.). In some examples, the processing device controls MEAD systems 200 located in a common space based on sensor data. The processing device may cause one MEAD system 200 to have a first operation (e.g., higher airflow, higher power to microwave generator 220) and cause other MEAD systems 200 in the same space to have a second operation (e.g., not operating, lower airflow, lower power to microwave generator 220) so that contaminants are destroyed without overworking all of the MEAD systems 200. The processing device may alternate which MEAD system 200 has the first operation to lessen wear-and-tear on a single MEAD system 200.

In some embodiments, the MEAD system 200 uses one or more products (e.g., multi-component filter 230, microwave generator 220, etc.) and/or one or more processes (e.g., using microwave energy generated by the microwave generator 220 to destroy contaminants trapped in the multi-component filter 230, controller 102 using sensor data from sensors 104 to control fan 240 and/or microwave generator 220 to destroy contaminants) relating to COVID-19 (e.g., destroying COVID-19 from the airflow) that is subject to an applicable Food and Drug Administration (FDA) and/or Environmental Protection Agency (EPA) approval for COVID-19 use.

In some embodiments, the microwave generator 220 provides microwave energy (e.g., radiofrequency microwave energy) through one or more waveguides (e.g., slot waveguide antennas) to the multi-component filter 230 to purify an airflow (e.g., air stream) containing contaminants (e.g., hazardous materials, organic vapors, etc.) and the multi-component filter 230 is regenerated without physical removal from the MEAD system 200.

The multi-component filter 230 may adsorb contaminants (e.g., organics) from contaminated airflow that passes through the multi-component filter 230 to purify the airflow. Saturation of the multi-component filter 230 (e.g., with contaminants) may eventually occur. Conventionally, a filter is replaced or the filter is removed for desorption via steam. The MEAD system 200 performs desorption of the multi-component filter 230 in situ by providing microwave energy (e.g., via a microwave generator 220 to a waveguide, such as slot waveguide antennas and while maintaining the microwave energy in the MEAD system 200 via microwave reflecting chamber).

The multi-component filter 230 is a good absorber of microwave energy (e.g., microwaves). The desorbed volatiles, which may not be in the same chemical form as they were when the adsorption occurred, are then removed via airflow (e.g., a sweep gas, operating the fan 240). The MEAD system 200 performs desorption (e.g., regeneration) without the multi-component filter 230 being removed for regeneration.

Quantum radiofrequency (RF) physics includes the phenomenon of resonant interaction with matter of electromagnetic radiation in the microwave and RF regions since atoms and molecules can absorb, and thus radiate, electromagnetic waves of various wavelengths. The rotational and vibrational frequencies of the electrons represent a frequency range. The electromagnetic frequency spectrum is usually divided into ultrasonic, microwave, and optical regions. In some embodiments, the microwave region is from 300 megahertz (MHz) to 300 gigahertz (GHz) and encompasses frequencies used for some communication equipment.

The term microwaves or microwave energy may be applied to a broad range of radiofrequency energies particularly with respect to the common heating and/or activating frequencies of about 915 MHz and about 2450 MHz. About 915 MHz is used in industrial heating applications and about 2450 MHz is the frequency of a common household microwave oven. In some embodiments, the MEAD system 200 uses microwave energy (e.g., microwaves) that is radiofrequency energies selected from the range of about 500 to 5000 MHz.

Microwaves lower the effective activation energy for chemical reactions since microwaves can act locally on a microscopic scale by exciting electrons of a group of specific atoms in contrast to normal global heating which raises the bulk temperature. The microscopic interaction is used by polar molecules whose electrons become locally excited leading to high chemical activity. The nonpolar molecules adjacent to such polar molecules are also affected but at a reduced extent. An example is the heating of polar water molecules in a common household microwave oven where the container is of nonpolar material, that is, microwave-passing, and stays relatively cool. In this sense microwaves are often referred to as a form of catalysis when applied to chemical reaction rates.

The MEAD system 200 provides an economically viable device for the microwave cleanup of impure air. The MEAD system 200 contains a multi-component filter 230 for adsorption of impurities that is regenerated in-place with radiofrequency energy in the microwave range by usage of a microwave generator 220 and one or more waveguides (e.g., slot antennas). The housing 210 forms a microwave cavity designed to reflect the microwaves leaving the waveguides into a center section containing the multi-component filter 230.

Microwaves (e.g., microwave energy) are a versatile form of energy that is applicable to enhance chemical reactions since the energy is locally applied by vibrational absorption by nonpolar molecules and does not produce plasma conditions. Reactions that proceed by free-radical mechanisms may be enhanced to higher rates (e.g., their initial equilibrium thermodynamics may be unfavorable).

The multi-component filter 230 may be an excellent microwave energy absorber and may include a wide range of polar impurities that readily interact with radiofrequency energy (e.g., in electron vibrational modes).

The multi-component filter 230 may be used under ambient temperature and pressure conditions. In some embodiments, the multi-component filter 230 includes a metal carbide (e.g., silicon carbide) as a microwave absorbing substrate to enhance catalytic processes.

The microwave excitation of the molecules of the multi-component filter 230, often referred to as microwave catalysis, excites constituents, such as impurities or contaminants including organics, which have been adsorbed on the internal pore surfaces of the multi-component filter 230 and produces a highly reactive condition. Further molecules from the carrier medium, such as a sweep gas (e.g., airflow), are in close proximity or within the surface boundary layer of the surface of the multi-component filter 230 through chemisorption, absorption, adsorption, or diffusion, and additional chemical reactions with these constituents may occur.

The desorption process potentially produces a wide range of chemical compounds since the microwave excited surface of the multi-component filter 230 and possibly the sweep gas molecules react with various decomposition products from the adsorbed constituents. Condensation of collected molecules from the sweep gas can be collected.

In some embodiments, the multi-component filter 230 includes a ceramic filter element that has a hollow space that includes a perforated tube (e.g., a centered perforated stainless steel tube). The space between the perforated tube and the ceramic filter may include pelletized filter material that removes impurities from the airflow. The multi-component filter 230 may be centered at a centerline in the inner volume of the housing 210 that reflects microwaves towards the centerline. One or more waveguides may be disposed in the housing 210 to direct microwaves towards the portions of the inner volume of the housing 210 that includes the multi-component filter 230. Airflow enters the housing 210 (e.g., via an inlet of the housing 210, via an open end of the housing 210), travels through the multi-component filter 230, is purified, and leaves the housing 210 (e.g., via an outlet of the housing).

When the multi-component filter 230 is saturated (e.g., as shown by measurements of impurities via sensors 104, such as a total hydrocarbon analyzer), the microwave generator 220 may be operated (e.g., by the controller 102) to regenerate the microwave generator 220.

In some embodiments, the microwave generator 220 provides microwave energy (e.g., microwaves) at about 2450 MHz. The MEAD system 200 may operate continuing cycles of adsorption (e.g., airflow without microwave energy) and desorption (e.g., microwave energy with or without airflow). In some embodiments, the microwave energy is employed at about 1000 watts.

In some embodiments, the MEAD system 200 has an elongated structural microwave cavity with inlet and exit regions configured to reflect microwaves onto a cavity-centered chamber (e.g., cylindrical chamber) that is designed for gas flow with a fixed multi-component filter 230 centered in the chamber. A waveguide (e.g., microwave slot antenna which may be located in the interior volume of the housing 210) may be used to radiate the cavity.

The inlet and exit regions of the housing 210 may be connections for airflow both for purifying the air and regeneration of the multi-component filter 230. The multi-component filter 230 may include at least two penetration depths measured with microwaves of about 2450 MHz. The frequency employed may affect the thickness of the multi-component filter 230 since the bed penetration by microwaves may be frequency dependent and further depend on the mass of the multi-component filter 230. For 2450 MHz microwaves, the penetration thickness (e.g., where the intensity of the RF energy has decreased by e⁻¹) of the multi-component filter 230 may be approximately one inch.

The waveguide (e.g., microwave slot antennas selected from the frequency range of 50 to 5000 MHz) may be capable of flexible operation (e.g., continuous source, pulsed source, cyclic source, periodic source, and combinations thereof). The size and spacing of the slots and the size of the waveguide (e.g., antenna) may be a function of microwave frequency.

In some embodiments, the MEAD system 200 is used to disinfect air (e.g., MEAD system 200 is used an air purification device, an air disinfection device, etc.). In some embodiments, the MEAD system 200 is used to detect a type or quantity of contaminant in the air (e.g., MEAD system 200 is used a contaminant detection device). A small amount of airflow may pass through the MEAD system 200 and sensor data from one or more sensors 104 (e.g., inlet sensor, off-gassing sensor, outlet sensor) can be used to determine whether there is a type or quantity of contaminant. The controller 102 may compare the sensor data (e.g., or differences between sensor data, such as difference between inlet sensor data and outlet sensor data) to threshold values and/or a reference data (e.g., a database of sensor data, a look-up table, etc.) to determine whether there is a type or quantity of contaminant in the air. Responsive to determining there is a type or quantity of contaminant in the air, the controller 102 may cause a corrective action (e.g., provide an alert, cause one or more other MEAD systems 200 to have a particular operation to disinfect the air, etc.).

FIGS. 3A-B are cross-sectional views of a MEAD system 300 (e.g., MEAD system 100 of FIG. 1, MEAD system 200 of FIGS. 2A and/or 2B), according to certain embodiments. Components of FIGS. 3A-B that have similar reference numbers as components in one or more of FIGS. 1-2 may have at least some of the same structure and/or functionality. FIG. 3A is a cross-sectional view length-wise of MEAD system 300 and FIG. 3B is a cross-sectional view width-wise of the MEAD system 300.

In some embodiments, the MEAD system 300 is a device (e.g., a stand-alone device, a device that can be installed in a system, a device that can be installed in ductwork, etc.). In some embodiments, the MEAD system 300 is substantially cylindrical.

In some embodiments, the MEAD system 300 includes a waveguide 324 that is routed through a central portion of the MEAD system 300 (e.g., along a longitudinal axis of the MEAD system 300, along a longitudinal axis of housing 310). In some embodiments, the waveguide 324 is cylindrical and slotted.

A first distal end of the MEAD system 300 may include a fan 340 disposed within a funnel 344 that is coupled to the housing 310. A second distal end of the MEAD system includes a microwave generator 320 coupled to the housing 310.

In some embodiments, the microwave generator 320 is coupled to the waveguide 324 via a magnetron tube 326. In some embodiments, the magnetron tube 326 has an outside perimeter (e.g., outer circumference) that is configured to fit within the inside diameter (inner circumference) of the waveguide 324. A housing 310 disposed around the waveguide 324. A multi-component filter 330 is disposed between the housing 310 and the waveguide 324. In some embodiments, the multi-component filter 330 is substantially a hollow cylinder. In some embodiments, the multi-component filter 330 includes two or more filter layers 332 (e.g., filter layers 332A-B). In some embodiments, the filter layers 332 contact each other. In some embodiments, the filter layers 332 are spaced apart. In some embodiments, filter layer 332A is a tubular microwave-reactive filter media. In some embodiments, filter layer 332B is a tubular HEPA filter with microwave reflective screening.

The microwave generator 320 generates microwave energy 322 that is channeled by the magnetron tube 326 into the waveguide 324 that directs the microwave energy 322 towards the multi-component filter 330. The fan 340 provides airflow 342 into the housing 310 to cool the microwave generator 320 and to pass through the multi-component filter 330 and then through the housing 310. Contaminants from the airflow 342 become trapped on the multi-component filter 330 and the microwave energy 322 causes the multi-component filter 330 to heat and/or activate to destroy the contaminants. In some embodiments, the microwave energy 322 is applied in a 360 degree pattern (e.g., around the cylindrical perimeter of the waveguide 324).

FIGS. 4A-B are cross-sectional views of a MEAD system 400 (e.g., MEAD system 200 of FIGS. 2A and/or B), according to certain embodiments. Components of FIGS. 4A-B that have similar reference numbers as components in one or more of FIGS. 1-3B may have at least some of the same structure and/or functionality. FIG. 4A is a cross-sectional view length-wise of MEAD system 400 and FIG. 4B is a cross-sectional view width-wise of the MEAD system 400.

In some embodiments, the MEAD system 400 is a device (e.g., a stand-alone device, a device that can be installed in a system, a device that can be installed in ductwork, etc.). In some embodiments, the MEAD system 400 is substantially a rectangular prism (e.g., opposing sides of the housing 410 are substantially parallel).

In some embodiments, the MEAD system 400 includes a waveguide 424 that is routed through the MEAD system 400 (e.g., parallel to a longitudinal axis of the MEAD system 400, parallel to a longitudinal axis of housing 410). In some embodiments, the waveguide 424 is a hollow rectangular prism and slotted (e.g., with slots directed towards the multi-component filter).

In some embodiments, a first distal end of the MEAD system 400 includes a fan 440 (e.g., disposed within a funnel that is coupled to the housing 410). A second distal end of the MEAD system includes a microwave generator 420 coupled to the housing 410.

In some embodiments, the microwave generator 420 is coupled to the waveguide 424 via a magnetron tube 426. In some embodiments, the magnetron tube 426 has an outside perimeter that is configured to fit within the inside diameter of the waveguide 424. A housing 410 disposed around the waveguide 424. A multi-component filter 430 is disposed between the housing 410 and the waveguide 424. In some embodiments, the multi-component filter 430 is substantially flat and is located between one side of the waveguide 424 and the housing 410. In some embodiments, the multi-component filter 430 includes two or more filter layers 432 (e.g., filter layers 432A-B). In some embodiments, the filter layers 432 contact each other. In some embodiments, the filter layers 432 are spaced apart.

The microwave generator 420 generates microwave energy 422 that is channeled by the magnetron tube 426 into the waveguide 424 that directs the microwave energy 422 towards the multi-component filter 430. The fan 440 may provide airflow 442 into the housing 410 to cool the microwave generator 420 and to pass through the multi-component filter 430 and then through the housing 410. Contaminants from the airflow 442 become trapped on the multi-component filter 430 and the microwave energy 422 causes the multi-component filter 430 to heat and/or activate to destroy the contaminants.

FIG. 5 illustrates data set generator 172 (e.g., data set generator 172 of FIG. 1) to create data sets for a machine learning model (e.g., model 190 of FIG. 1) associated with one or more MEAD systems, according to certain embodiments. In some embodiments, data set generator 172 is part of server machine 170 of FIG. 1.

Data set generator 172 creates data sets for a machine learning model (e.g., model 190 of FIG. 1). Data set generator 172 creates data sets using historical sensor data 144 and historical performance data 154. System 500 of FIG. 5 shows data set generator 172, data inputs 510, and target output 520.

In some embodiments, data set generator 172 generates a data set (e.g., training set, validating set, testing set) that includes one or more data inputs 510 (e.g., training input, validating input, testing input) and one or more target outputs 520 that correspond to the data inputs 510. The data set also includes mapping data that maps the data inputs 510 to the target outputs 520. Data inputs 510 are also referred to as “features,” “attributes,” or “information.” In some embodiments, data set generator 172 provides the data set to the training engine 182, validating engine 184, or testing engine 186, where the data set is used to train, validate, or test the machine learning model 190. Some embodiments of generating a training set are further described with respect to FIG. 7A.

In some embodiments, data set generator 172 generates the data input 510 and target output 520. In some embodiments, data inputs 510 include one or more sets of historical sensor data 144. Each instance of historical sensor data 144 includes one or more of sensor data from one or more types of sensors, combination of sensor data from one or more types of sensors, patterns from sensor data from one or more types of sensors, etc.

In some embodiments, data set generator 172 generates a first data input corresponding to a first set of historical sensor data 144A to train, validate, or test a first machine learning model and the data set generator 172 generates a second data input corresponding to a second set of historical sensor data 144B to train, validate, or test a second machine learning model.

In some embodiments, the data set generator 172 discretizes (e.g., segments) one or more of the data input 510 or the target output 520 (e.g., to use in classification algorithms for regression problems). Discretization (e.g., segmentation via a sliding window) of the data input 510 or target output 520 transforms continuous values of variables into discrete values. In some embodiments, the discrete values for the data input 510 indicate discrete historical sensor data 144 to obtain a target output 520 (e.g., discrete performance data 154).

Data inputs 510 and target outputs 520 to train, validate, or test a machine learning model include information for a particular location (e.g., region, city, building, room, etc.). In some examples, historical sensor data 144 and historical performance data 154 are for the same location and/or MEAD system.

In some embodiments, the information used to train the machine learning model is from specific types and/or groups of MEAD systems having specific characteristics (e.g., same or similar structure, same or similar multi-component filters, etc.) and allow the trained machine learning model to determine outcomes for same or similar types and/or groups of MEAD systems having same or similar specific characteristics based on current sensor data 146.

In some embodiments, subsequent to generating a data set and training, validating, or testing a machine learning model 190 using the data set, the machine learning model 190 is further trained, validated, or tested (e.g., current performance data 156 of FIG. 1) or adjusted (e.g., adjusting weights associated with input data of the machine learning model 190, such as connection weights in a neural network).

FIG. 6 is a block diagram illustrating a system 600 (e.g., predictive system 130 of FIG. 1) for generating predictive data 160 associated with one or more MEAD systems, according to certain embodiments. The system 600 is used to determine predictive data 160 (e.g., via model 190 of FIG. 1) to cause a corrective action associated with a MEAD system (e.g., MEAD system 100 of FIG. 1, MEAD system 200 of FIGS. 2A and/or 2B, MEAD system 300 of FIGS. 3A-B, MEAD system 400 of FIGS. 4A-B, etc.).

At block 610, the system 600 performs data partitioning (e.g., via data set generator 172 of server machine 170 of FIG. 1) of the historical data (e.g., historical sensor data 144 and historical performance data 154 of FIG. 1) to generate the training set 602, validation set 604, and testing set 606. In some examples, the training set is 60% of the historical data, the validation set is 20% of the historical data, and the testing set is 20% of the historical data. The system 600 generates a plurality of sets of features for each of the training set, the validation set, and the testing set. In some examples, if the historical data includes features derived from sensor data from 20 sensors (e.g., sensors 104 of FIG. 1) and 100 iterations (e.g., iterations associated with generating microwave energy, actuating the fan, etc. that each correspond to the sensor data from the 20 sensors), a first set of features is sensors 1-10, a second set of features is sensors 11-20, the training set is iterations 1-60, the validation set is iterations 61-80, and the testing set is iterations 81-100. In this example, the first set of features of the training set would be sensor data from sensors 1-10 for iterations 1-60.

At block 612, the system 600 performs model training (e.g., via training engine 182 of FIG. 1) using the training set 602. In some embodiments, the system 600 trains multiple models using multiple sets of features of the training set 602 (e.g., a first set of features of the training set 602, a second set of features of the training set 602, etc.). For example, system 600 trains a machine learning model to generate a first trained machine learning model using the first set of features in the training set (e.g., sensor data from sensors 1-10 for iterations 1-60) and to generate a second trained machine learning model using the second set of features in the training set (e.g., sensor data from sensors 11-20 for iterations 1-60). In some embodiments, the first trained machine learning model and the second trained machine learning model are combined to generate a third trained machine learning model (e.g., a better predictor than the first or the second trained machine learning model on its own in some embodiments). In some embodiments, sets of features used in comparing models overlap (e.g., first set of features being sensor data from sensors 1-15 and second set of features being sensors 5-20). In some embodiments, hundreds of models are generated including models with various permutations of features and combinations of models.

At block 614, the system 600 performs model validation (e.g., via validation engine 184 of FIG. 1) using the validation set 604. The system 600 validates each of the trained models using a corresponding set of features of the validation set 604. For example, system 600 validates the first trained machine learning model using the first set of features in the validation set (e.g., sensor data from sensors 1-10 for iterations 61-80) and the second trained machine learning model using the second set of features in the validation set (e.g., sensor data from sensors 11-20 for iterations 61-80). In some embodiments, the system 600 validates hundreds of models (e.g., models with various permutations of features, combinations of models, etc.) generated at block 612. At block 614, the system 600 determines an accuracy of each of the one or more trained models (e.g., via model validation) and determines whether one or more of the trained models has an accuracy that meets a threshold accuracy. Responsive to determining that none of the trained models has an accuracy that meets a threshold accuracy, flow returns to block 612 where the system 600 performs model training using different sets of features of the training set. Responsive to determining that one or more of the trained models has an accuracy that meets a threshold accuracy, flow continues to block 616. The system 600 discards the trained machine learning models that have an accuracy that is below the threshold accuracy (e.g., based on the validation set).

At block 616, the system 600 performs model selection (e.g., via selection engine 185 of FIG. 1) to determine which of the one or more trained models that meet the threshold accuracy has the highest accuracy (e.g., the selected model 608, based on the validating of block 614). Responsive to determining that two or more of the trained models that meet the threshold accuracy have the same accuracy, flow returns to block 612 where the system 600 performs model training using further refined training sets corresponding to further refined sets of features for determining a trained model that has the highest accuracy.

At block 618, the system 600 performs model testing (e.g., via testing engine 186 of FIG. 1) using the testing set 606 to test the selected model 608. The system 600 tests, using the first set of features in the testing set (e.g., sensor data from sensors 1-10 for iterations 81-100), the first trained machine learning model to determine the first trained machine learning model meets a threshold accuracy (e.g., based on the first set of features of the testing set 606). Responsive to accuracy of the selected model 608 not meeting the threshold accuracy (e.g., the selected model 608 is overly fit to the training set 602 and/or validation set 604 and is not applicable to other data sets such as the testing set 606), flow continues to block 612 where the system 600 performs model training (e.g., retraining) using different training sets corresponding to different sets of features (e.g., sensor data from different sensors). Responsive to determining that the selected model 608 has an accuracy that meets a threshold accuracy based on the testing set 606, flow continues to block 620. In at least block 612, the model learns patterns in the historical data to make predictions and in block 618, the system 600 applies the model on the remaining data (e.g., testing set 606) to test the predictions.

At block 620, system 600 uses the trained model (e.g., selected model 608) to receive current sensor data 146 and determines (e.g., extracts), from the output of the trained model, predictive data 160 to perform corrective actions associated with the MEAD system 100. In some embodiments, the current sensor data 146 corresponds to the same types of features in the historical sensor data 144. In some embodiments, the current sensor data 146 corresponds to a same type of features as a subset of the types of features in historical sensor data that are used to train the selected model 608.

In some embodiments, current data is received. In some embodiments, current data includes current performance data 156. The model 608 is re-trained based on the current data. In some embodiments, a new model is trained based on the current data and the current sensor data 146.

In some embodiments, one or more of the operations 610-620 occur in various orders and/or with other operations not presented and described herein. In some embodiments, one or more of operations 610-620 are not be performed. For example, in some embodiments, one or more of data partitioning of block 610, model validation of block 614, model selection of block 616, and/or model testing of block 618 are not performed.

FIGS. 7A-E are flow diagrams of methods 700A-E associated with one or more MEAD systems, according to certain embodiments. In some embodiments, methods 700A-E are performed by processing logic that includes hardware (e.g., circuitry, dedicated logic, programmable logic, microcode, processing device, etc.), software (such as instructions run on a processing device, a general purpose computer system, or a dedicated machine), firmware, microcode, or a combination thereof. In some embodiment, one or more of methods 700A-E are performed, at least in part, by predictive system 130, client device 136, and/or controller 102 of MEAD system 100 of FIG. 1. In some embodiments, method 700A is performed, at least in part, by predictive system 130 (e.g., server machine 170 and data set generator 172 of FIG. 1, data set generator 172 of FIG. 5). In some embodiments, predictive system 130 uses method 700A to generate a data set to at least one of train, validate, or test a machine learning model. In some embodiments, method 700C is performed by controller 102 and/or predictive system 130. In some embodiments, method 700C is performed by server machine 180 (e.g., training engine 182, etc.). In some embodiments, method 700D is performed by predictive server 112 (e.g., predictive component 114). In some embodiments, a non-transitory machine-readable storage medium stores instructions that when executed by a processing device (e.g., of predictive system 130, of server machine 180, of predictive server 112, controller 102, etc.), cause the processing device to perform one or more of methods 700A-E. In some embodiments, any of the methods described herein are performed by a server, by a client device 136, and/or a controller 102 of a MEAD system 100.

For simplicity of explanation, methods 700A-E are depicted and described as a series of operations. However, operations in accordance with this disclosure can occur in various orders and/or concurrently and with other operations not presented and described herein. Furthermore, in some embodiments, not all illustrated operations are performed to implement methods 700A-E in accordance with the disclosed subject matter. In addition, those skilled in the art will understand and appreciate that methods 700A-E could alternatively be represented as a series of interrelated states via a state diagram or events.

FIG. 7A is a flow diagram of a method 700A for generating a data set for a machine learning model for generating predictive data (e.g., predictive data 160 of FIG. 1), according to certain embodiments.

Referring to FIG. 7A, in some embodiments, at block 701 the processing logic implementing method 700A initializes a training set T to an empty set.

At block 702, processing logic generates first data input (e.g., first training input, first validating input, first testing input, etc.) that includes sensor data (e.g., historical sensor data 144 of FIGS. 1, 5, and/or 6). In some embodiments, the first data input includes a first set of features for types of sensor data and a second data input includes a second set of features for types of sensor data (e.g., as described with respect to FIG. 5). In some embodiments, the historical sensor data includes flow rate, pressure, temperature, power usage, off-gassing data, and/or the like.

At block 703, processing logic generates a first target output for one or more of the data inputs (e.g., first data input). In some embodiments, the first target output is historical performance data (e.g., historical performance data 154 of FIGS. 1, 5, and/or 6). In some embodiments, the historical performance data includes flow rates, off-gassing data, pressure data, quality data of the air entering the MEAD system, contaminants in the air entering the MEAD system, and/or the like. In some embodiments, the historical performance data is associated with performance of a corrective action (e.g., iterations of generating microwave energy, iterations of operating the fan, schedule of maintenance, etc.).

At block 704, processing logic optionally generates mapping data that is indicative of an input/output mapping. The input/output mapping (or mapping data) refers to the data input (e.g., one or more of the data inputs described herein), the target output for the data input (e.g., where the target output identifies historical performance data 154), and an association between the data input(s) and the target output.

At block 705, processing logic adds the mapping data generated at block 704 to data set T.

At block 706, processing logic branches based on whether data set T is sufficient for at least one of training, validating, and/or testing machine learning model 190. If so, execution proceeds to block 707, otherwise, execution continues back at block 702. It should be noted that in some embodiments, the sufficiency of data set T is determined based simply on the number of input/output mappings in the data set, while in some other implementations, the sufficiency of data set T is determined based on one or more other criteria (e.g., a measure of diversity of the data examples, accuracy, etc.) in addition to, or instead of, the number of input/output mappings.

At block 707, processing logic provides data set T (e.g., to server machine 180) to train, validate, and/or test machine learning model 190. In some embodiments, data set T is a training set and is provided to training engine 182 of server machine 180 to perform the training. In some embodiments, data set T is a validation set and is provided to validation engine 184 of server machine 180 to perform the validating. In some embodiments, data set T is a testing set and is provided to testing engine 186 of server machine 180 to perform the testing. In the case of a neural network, for example, input values of a given input/output mapping (e.g., numerical values associated with data inputs 510) are input to the neural network, and output values (e.g., numerical values associated with target outputs 520) of the input/output mapping are stored in the output nodes of the neural network. The connection weights in the neural network are then adjusted in accordance with a learning algorithm (e.g., back propagation, etc.), and the procedure is repeated for the other input/output mappings in data set T. After block 707, machine learning model (e.g., machine learning model 190) can be at least one of trained using training engine 182 of server machine 180, validated using validating engine 184 of server machine 180, or tested using testing engine 186 of server machine 180. The trained machine learning model is implemented by predictive component 114 (of predictive server 112) to generate predictive data 160 for performing corrective action associated with a MEAD system.

FIG. 7B is a flow diagram of a method 700B associated with control of a MEAD system (e.g., MEAD system 100 of FIG. 1, MEAD system 200 of FIGS. 2A and/or 2B, MEAD system 300 of FIGS. 3A-B, MEAD system 400 of FIGS. 4A-B), according to certain embodiments. In some embodiments, one or more operations of method 700A are performed by predictive system 130, predictive server 132, client device, or controller 102 of FIG. 1.

Referring to FIG. 7B, at block 720 processing logic receives sensor data associated with a microwave generator of a MEAD system intermittently generating microwave energy to destroy contaminants from airflow collected on the multi-component filter. In some embodiments, the sensor data is from one or more sensors located proximate the multi-component filter, an inlet, and/or an outlet of a MEAD system. In some embodiments, the sensor data is associated with properties of the off gas resulting from at least a portion of the multi-component filter being heated and/or activated by microwave energy to destroy (e.g., off gas) contaminants trapped on the multi-component filter (e.g., responsive to contaminated airflow passing through the multi-component filter). The properties of the off gas may include composition of the off gas, amount of off gas, length of time of the off gassing, etc.

In some embodiments, the sensor data is a full feedback loop of sensors (e.g., sensor taking readings at an entry point of the MEAD system, a sensor taking readings of the off gas, and a sensor taking readings at the exit). In some embodiments, the sensor data is aggregated to generate air content data and/or delta elimination data (e.g., indicative of a quantity of the contaminants that was destroyed). In some embodiments, the sensor data (e.g., air content data) is stored in a database. The sensor data can be retrieved (e.g., from the database) to train a machine learning model (e.g., see FIG. 7C) and/or to be input into a trained machine learning model to determine a corrective action (e.g., see FIG. 7D). Pattern recognition and/or machine learning may be used to make predictions about type or quantity of contaminants (e.g., how much bad virus) is in the air to update one or more MEAD systems. Machine learning can be used to perform regional and cluster updates to update multiple MEAD systems. If a MEAD system (e.g., a single MEAD unit) has sensor data that indicates that there is a type of contaminant (e.g., virus) that is being destroyed by the MEAD system, other MEAD systems in the region (e.g., neighborhood, town, etc.) can be updated (e.g., via instructions from the MEAD system, a client device, a gateway device, a server device) to operate more aggressively as a preventative measure.

At block 722, processing logic causes, based on the sensor data, performance of a corrective action. In some embodiments, the corrective action is generating or updating a schedule for generating microwave energy and/or operating the fan responsive to determining how the sensor data compares to threshold data. In some examples, responsive to an amount of off gas being above a threshold amount, a flow rate being below a threshold rate, a particular composition of off gas, and/or the like, the corrective action may include generating microwave energy more often, generating microwave energy for longer periods of time, operating the fan more often, and/or operating the fan for longer periods of time. In some examples, responsive to an amount of off gas being below a threshold amount, flow rate being above a threshold rate, a particular composition of off gas, and/or the like, the corrective action may include generating microwave energy less often and/or operating the fan less often. In some embodiments, the corrective action may include causing replacement of a component of the MEAD system and/or preventative maintenance. In some embodiments, the corrective action may include interrupting operation (e.g., generating of microwave energy, operating the fan) of the MEAD system. In some embodiments, the performance of the corrective action includes generating or updating a schedule for collection and/or transmission of data, etc.

In some embodiments, the corrective action includes providing an alert (e.g., to client device 136 of FIG. 1, to a user device, etc.). In some examples, the processing logic determines information based on the first sensor data. In some embodiments, the alert includes information (e.g., determined based on the sensor data, that is predictive data determined based on the sensor data, etc.) associated with one or more of quality of incoming air, confirmation of destruction of the contaminants, performance (e.g., efficacy) of the MEAD system, and/or the like.

In some embodiments, blocks 720-722 are repeated to one or more of cause performance of updated corrective actions, stop performing a corrective action, update a schedule of operations, and/or the like.

In some embodiments, the corrective action of block 722 of FIG. 7B is determined by providing input of sensor data of block 720 of FIG. 7B to a trained machine learning model (e.g., see FIG. 1, FIGS. 5-6, and FIG. 7D).

FIG. 7C is a method for training a machine learning model (e.g., model 190 of FIG. 1) for determining predictive data (e.g., predictive data 160 of FIG. 1) to perform a corrective action associated with a MEAD system.

Referring to FIG. 7C, at block 740 of method 700C, the processing logic receives sets of historical sensor data (e.g., historical sensor data 144 of FIG. 1) associated with one or more MEAD systems. In some embodiments, the sensor data is collected over time from sensors of different MEAD systems. In some embodiments, the sensor data is associated with different off gassing of contaminants from the multi-component filters via microwave energy and airflow.

At block 742, the processing logic receives sets of historical performance data (e.g., historical performance data 154 of FIG. 1) associated with the one or more MEAD systems. Each of the sets of the historical performance data corresponds to a respective set of historical sensor data of the sets of historical sensor data. In some embodiments, the historical performance data is associated with quality of airflow (e.g., known contaminants in the airflow) that is provided to the MEAD system, operation of the microwave generator, operation of the fan, quality of airflow that exits the MEAD system, etc.

In some embodiments, the historical performance data includes resulting sensor data after performing a corrective action. In some embodiments, the historical performance data includes pressure data, air flow rates, off gas data, power used, and/or the like responsive to a corrective action associated with operating the MEAD system (e.g., generating microwave energy, operating the fan, and/or the like). In some embodiments, the historical performance data includes information associated with the corrective action performed, such as iterations of microwave energy generation, iterations of fan operation, schedule of microwave energy generation, schedule of fan operation, and/or the like.

In some examples, airflow with known contaminants (e.g., historical performance data) is provided to the MEAD system and resulting off gassing data (e.g., historical sensor data) is obtained. The machine learning model is trained to relate the off gassing data of the MEAD system to known contaminants in airflow entering the MEAD system. The trained machine learning model can then be provided current sensor data associated with current off gassing and predict the contaminants in the airflow entering the MEAD system.

At block 744, the processing logic trains a machine learning model using data input including the sets of historical sensor data and target output including the historical performance data to generate a trained machine learning model. The trained machine learning model is capable of generating outputs indicative of predictive data (e.g., predictive data 160) to cause performance of one or more corrective actions (e.g., based on current sensor data) associated with one or more operating modules of a MEAD system.

FIG. 7D is a method 700D for using a trained machine learning model (e.g., model 190 of FIG. 1) for determining predictive data to cause performance of a corrective action associated with a MEAD system.

Referring to FIG. 7D, at block 760 of method 700C, the processing logic receives sets of sensor data (e.g., current sensor data 146 of FIG. 1) associated with a MEAD system. In some embodiments, the sensor data is associated with one or more of fan operation, microwave energy generation, off gassing, etc.

At block 762, the processing logic provides the sets of sensor data as input to a trained machine learning model (e.g., the trained machine learning model of block 744 of FIG. 7C).

At block 764, the processing logic obtains, from the trained machine learning model, one or more outputs indicative of predictive data. In some embodiments, the predictive data is associated with predicted performance data resulting from performance of one or more corrective actions, lack of performance of a corrective action, a schedule of performing corrective actions, type of corrective actions (e.g., iterations of microwave energy generation and/or fan operation), and/or the like. In some embodiments, the predictive data is associated with quality of airflow (e.g., type and/or quantity of contaminants) entering the MEAD system.

At block 766, the processing logic causes, based on the one or more outputs (e.g., predictive data), performance of a corrective action associated with the MEAD system.

In some embodiments, the corrective action corresponds to operations (e.g., iterations of generation of microwave energy and/or fan operation), a replacement of a component (e.g., a pre-filter), a repair, an update to a schedule (e.g., update schedule of microwave energy generation and/or fan operation), and/or the like.

At block 768, processing logic receives performance data (e.g., current performance data 156 of FIG. 1) associated with the MEAD system (e.g., associated with the sets of sensor data from block 720). In some embodiments, the performance data is associated with operation of the MEAD system after the performance of the corrective action (e.g., pressure data after performing a microwave energy generation). In some embodiments, the performance data is associated with quality of airflow (e.g., type and/or quantity of contaminants) entering the MEAD system. In some embodiments, the performance data received is different from the predicted data and in some embodiments, the performance data is substantially similar to the predicted data.

At block 770, processing logic causes the trained machine learning model to be further trained (e.g., re-trained) with data input including the sets of sensor data (e.g., from block 760) and target output including the performance data (e.g., from block 768).

In some embodiments, blocks 760-764 are repeated until the one or more outputs (e.g., predictive data) indicates that no further corrective actions are to be performed (e.g., predictive data indicates predictive performance data that is the same as the schedule for generating microwave energy and/or operating the fan without performing a corrective action).

FIG. 7E is a flow diagram of a method 700E associated with control of a MEAD system (e.g., MEAD system 100 of FIG. 1, MEAD system 200 of FIGS. 2A and/or 2B, MEAD system 300 of FIGS. 3A-B, MEAD system 400 of FIGS. 4A-B), according to certain embodiments. In some embodiments, one or more operations of method 700E are performed by predictive system 130, predictive server 132, client device, or controller 102 of FIG. 1.

Referring to FIG. 7E, at block 780 processing logic identifies a schedule to operate a MEAD system. The schedule may indicate when the MEAD system is to generate microwave energy at particular power settings, when the MEAD system is to provide particular airflow via the fan, etc. In some embodiments, the schedule is a default schedule (e.g., a schedule that all MEAD systems use). In some embodiments, the schedule is a schedule generated for MEAD systems in an area (e.g., region, building, etc.) based on sensor data (e.g., collected by MEAD systems, retrieved from third parties, etc.). In some embodiments, the schedule is generated based on sensor data and/or user input of other MEAD systems.

At block 782, processing logic causes, based on the schedule, intermittent generation of microwave energy (e.g., generation of microwave energy at particular times, for particular duration of time, at particular power levels, at particular intervals, etc.) by a microwave generator of the MEAD system. In some embodiments, the processing logic causes, based on the schedule, intermittent generation of airflow (e.g., generation of airflow at particular times, for particular duration of time, at particular flow rates, at particular intervals, etc.) by the fan of the MEAD system.

At block 784, processing logic receives sensor data or user input. The sensor data may be received from sensors located at an inlet, an off-gas location, and/or an outlet. The user input may be associated with the airflow, the power level of the microwave energy generation, overriding the schedule, etc. In some embodiments, the sensor data and/or user input is associated with the MEAD system (e.g., via sensors of the MEAD system and user input to control the MEAD system). In some embodiments, the sensor data and/or user input is associated with one or more MEAD systems (e.g., in the same region, in the same building, etc.).

At block 786, processing logic determines whether the sensor data or the user input matches the schedule. In some examples, the schedule indicates that there is to be less than a threshold value of sensor data from the sensor proximate the off-gassing. In some examples, the schedule indicates there is to be less than a threshold distance value of a difference in sensor data between a sensor proximate the inlet and a sensor proximate the outlet. In some examples, the schedule indicates a pattern of user input.

Responsive to the sensor data and user input matching the schedule, flow returns to block 782 to continue using the same schedule. Responsive to the sensor data or user input not matching the schedule, flow continues to block 788.

At block 788, processing logic updates, based on the sensor data or the user input, the schedule to operate the MEAD system. In some examples, responsive to determining the sensor data meets a threshold value or the difference in sensor data meets a threshold distance value (e.g., indicating more than a threshold amount of contaminants), the processing device updates the operation of the MEAD system (e.g., increases power to the microwave generator, increases the duration of operation of the microwave generator, increases how often the microwave generator runs, etc.). In some examples, responsive to determining the user input does not match the patterns in the schedule, the processing device causes the schedule to be updated based on the new user input (e.g., the new pattern of user input).

FIG. 8 is a block diagram illustrating a computer system 800, according to certain embodiments. In some embodiments, the computer system 800 is one or more of client device 120, predictive system 130, server machine 170, server machine 180, predictive server 112, controller of the MEAD system (controller 102 of MEAD system 100), etc. In some embodiments, the processor 802 is the controller of the MEAD system (controller 102 of MEAD system 100).

In some embodiments, computer system 800 is connected (e.g., via a network, such as a Local Area Network (LAN), an intranet, an extranet, or the Internet) to other computer systems. In some embodiments, computer system 800 operates in the capacity of a server or a client computer in a client-server environment, or as a peer computer in a peer-to-peer or distributed network environment. In some embodiments, computer system 800 is provided by a personal computer (PC), a tablet PC, a Set-Top Box (STB), a Personal Digital Assistant (PDA), a cellular telephone, a web appliance, a server, a network router, switch or bridge, or any device capable of executing a set of instructions (sequential or otherwise) that specify actions to be taken by that device. Further, the term “computer” shall include any collection of computers that individually or jointly execute a set (or multiple sets) of instructions to perform any one or more of the methods described herein (e.g., one or more of methods 700A-E of FIGS. 7A-E, etc.).

In a further aspect, the computer system 800 includes a processing device 802, a volatile memory 804 (e.g., Random Access Memory (RAM)), a non-volatile memory 806 (e.g., Read-Only Memory (ROM) or Electrically-Erasable Programmable ROM (EEPROM)), and a data storage device 816, which communicate with each other via a bus 808.

In some embodiments, processing device 802 is provided by one or more processors such as a general purpose processor (such as, for example, a Complex Instruction Set Computing (CISC) microprocessor, a Reduced Instruction Set Computing (RISC) microprocessor, a Very Long Instruction Word (VLIW) microprocessor, a microprocessor implementing other types of instruction sets, or a microprocessor implementing a combination of types of instruction sets) or a specialized processor (such as, for example, an Application Specific Integrated Circuit (ASIC), a Field Programmable Gate Array (FPGA), a Digital Signal Processor (DSP), or a network processor).

In some embodiments, computer system 800 further includes a network interface device 822 (e.g., coupled to network 874). In some embodiments, computer system 800 also includes a video display unit 810 (e.g., an LCD), an alphanumeric input device 812 (e.g., a keyboard), a cursor control device 814 (e.g., a mouse), and a signal generation device 820.

In some implementations, data storage device 816 includes a non-transitory computer-readable storage medium 824 on which store instructions 826 encoding any one or more of the methods or functions described herein, including instructions for implementing methods described herein.

In some embodiments, instructions 826 also reside, completely or partially, within volatile memory 804 and/or within processing device 802 during execution thereof by computer system 800, hence, in some embodiments, volatile memory 804 and processing device 802 also constitute machine-readable storage media.

While computer-readable storage medium 824 is shown in the illustrative examples as a single medium, the term “computer-readable storage medium” shall include a single medium or multiple media (e.g., a centralized or distributed database, and/or associated caches and servers) that store the one or more sets of executable instructions. The term “computer-readable storage medium” shall also include any tangible medium that is capable of storing or encoding a set of instructions for execution by a computer that cause the computer to perform any one or more of the methods described herein. The term “computer-readable storage medium” shall include, but not be limited to, solid-state memories, optical media, and magnetic media.

In some embodiments, the methods, components, and features described herein are implemented by discrete hardware components or are integrated in the functionality of other hardware components such as ASICS, FPGAs, DSPs or similar devices. In some embodiments, the methods, components, and features are implemented by firmware modules or functional circuitry within hardware devices. In some embodiments, the methods, components, and features are implemented in any combination of hardware devices and computer program components, or in computer programs.

Unless specifically stated otherwise, terms such as “identifying,” “receiving,” “causing,” “training,” “generating,” “providing,” “obtaining,” “interrupting,” “determining,” “transmitting,” or the like, refer to actions and processes performed or implemented by computer systems that manipulates and transforms data represented as physical (electronic) quantities within the computer system registers and memories into other data similarly represented as physical quantities within the computer system memories or registers or other such information storage, transmission or display devices. In some embodiments, the terms “first,” “second,” “third,” “fourth,” etc. as used herein are meant as labels to distinguish among different elements and do not have an ordinal meaning according to their numerical designation.

Examples described herein also relate to an apparatus for performing the methods described herein. In some embodiments, this apparatus is specially constructed for performing the methods described herein, or includes a general purpose computer system selectively programmed by a computer program stored in the computer system. Such a computer program is stored in a computer-readable tangible storage medium.

Some of the methods and illustrative examples described herein are not inherently related to any particular computer or other apparatus. In some embodiments, various general purpose systems are used in accordance with the teachings described herein. In some embodiments, a more specialized apparatus is constructed to perform methods described herein and/or each of their individual functions, routines, subroutines, or operations. Examples of the structure for a variety of these systems are set forth in the description above.

The above description is intended to be illustrative, and not restrictive. Although the present disclosure has been described with references to specific illustrative examples and implementations, it will be recognized that the present disclosure is not limited to the examples and implementations described. The scope of the disclosure should be determined with reference to the following claims, along with the full scope of equivalents to which the claims are entitled.

The preceding description sets forth numerous specific details such as examples of specific systems, components, methods, and so forth in order to provide a good understanding of several embodiments of the present disclosure. It will be apparent to one skilled in the art, however, that at least some embodiments of the present disclosure may be practiced without these specific details. In other instances, well-known components or methods are not described in detail or are presented in simple block diagram format in order to avoid unnecessarily obscuring the present disclosure. Thus, the specific details set forth are merely exemplary. Particular implementations may vary from these exemplary details and still be contemplated to be within the scope of the present disclosure.

The terms “over,” “under,” “between,” “disposed on,” and “on” as used herein refer to a relative position of one material layer or component with respect to other layers or components. For example, one layer disposed on, over, or under another layer may be directly in contact with the other layer or may have one or more intervening layers. Moreover, one layer disposed between two layers may be directly in contact with the two layers or may have one or more intervening layers. Similarly, unless explicitly stated otherwise, one feature disposed between two features may be in direct contact with the adjacent features or may have one or more intervening layers.

The words “example” or “exemplary” are used herein to mean serving as an example, instance or illustration. Any aspect or design described herein as “example’ or “exemplary” is not necessarily to be construed as preferred or advantageous over other aspects or designs. Rather, use of the words “example” or “exemplary” is intended to present concepts in a concrete fashion.

Reference throughout this specification to “one embodiment,” “an embodiment,” or “some embodiments” means that a particular feature, structure, or characteristic described in connection with the embodiment is included in at least one embodiment. Thus, the appearances of the phrase “in one embodiment,” “in an embodiment,” or “in some embodiments” in various places throughout this specification are not necessarily all referring to the same embodiment. In addition, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Also, the terms “first,” “second,” “third,” “fourth,” etc. as used herein are meant as labels to distinguish among different elements and can not necessarily have an ordinal meaning according to their numerical designation. When the term “about,” “substantially,” or “approximately” is used herein, this is intended to mean that the nominal value presented is precise within ±10%.

Although the operations of the methods herein are shown and described in a particular order, the order of operations of each method may be altered so that certain operations may be performed in an inverse order so that certain operations may be performed, at least in part, concurrently with other operations. In another embodiment, instructions or sub-operations of distinct operations may be in an intermittent and/or alternating manner.

It is understood that the above description is intended to be illustrative, and not restrictive. Many other embodiments will be apparent to those of skill in the art upon reading and understanding the above description. The scope of the disclosure should, therefore, be determined with reference to the appended claims, along with the full scope of equivalents to which such claims are entitled.

What is claimed is:

1. A method comprising:
identifying, by a processing device, a schedule to operate a microwave enhanced air disinfection (MEAD) system; and
causing, based on the schedule, intermittent generation of microwave energy by a microwave generator of the MEAD system, wherein a multi-component filter disposed in a housing of the MEAD system is configured to collect contaminants from airflow through the housing, and wherein at least a portion of the contaminants from the airflow is to be destroyed at least one of directly or indirectly via the microwave energy.

2. The method of claim 1, wherein one or more of:
the schedule is based on sensor data associated with one or more MEAD systems;
the schedule is based on first sensor data received from a sensor of the MEAD system; or
the schedule is based on user input received via the MEAD system.

3. The method of claim 1 further comprising:
receiving first sensor data associated with the microwave generator of the MEAD system intermittently generating the microwave energy to destroy the at least a portion of the contaminants from the airflow; and
causing, based on the first sensor data, performance of a corrective action associated with the MEAD system.

4. The method of claim 3, wherein the first sensor data received from one or more of:
a sensor disposed proximate an inlet of the MEAD system;
a sensor disposed proximate off-gassing of the contaminants in the MEAD system; or
a sensor disposed proximate an outlet of the MEAD system.

5. The method of claim 3 further comprising:
receiving second sensor data associated with the MEAD system; and
causing, based on the second sensor data, the performance of the corrective action to cease.

6. The method of claim 1 further comprising:
receiving historical sensor data associated with one or more MEAD systems;
receiving historical performance data associated with the one or more MEAD systems; and
training a machine learning model with data input comprising the historical sensor data and target data comprising the historical performance data to generate a trained machine learning model, the trained machine learning model capable of generating one or more outputs indicative of predictive data for performing one or more corrective actions.

7. The method of claim 6, wherein:
the historical sensor data is associated with corresponding off gas of the one or more MEAD systems during generation of corresponding microwave energy to activate a corresponding multi-component filter; and
the historical performance data is associated with one or more of quality of historical airflow or operation of the one or more MEAD systems.

8. The method of claim 3 further comprising:
providing the first sensor data to a trained machine learning model; and
obtaining, from the trained machine learning model, one or more outputs indicative of predictive data, wherein the causing of the performance of the corrective action is based on the predictive data.

9. The method of claim 3, wherein the corrective action comprises one or more of:
updating the schedule to operate the MEAD system;
causing the microwave generator to generate the microwave energy for a first quantity of time;
causing a fan of the MEAD system to provide the airflow through the MEAD system for a second quantity of time;
causing one or more portions of the multi-component filter to be replaced;
interrupting the generation of the microwave energy; or
causing an alert to be provided.

10. The method of claim 3 further comprising:
determining, based on the first sensor data, information associated with one or more of:
quality of incoming air;
confirmation of destruction of the at least a portion of the contaminants; or
performance of the MEAD system.

11. A non-transitory machine-readable storage medium storing instructions which, when executed cause a processing device to perform operations comprising:
identifying a schedule to operate a microwave enhanced air disinfection (MEAD) system; and
causing, based on the schedule, intermittent generation of microwave energy by a microwave generator of the MEAD system, wherein a multi-component filter disposed in a housing of the MEAD system is configured to collect contaminants from airflow through the housing, and wherein at least a portion of the contaminants from the airflow is to be destroyed at least one of directly or indirectly via the microwave energy.

12. The non-transitory machine-readable storage medium of claim 11, wherein one or more of:
the schedule is based on sensor data associated with one or more MEAD systems;
the schedule is based on first sensor data received from a sensor of the MEAD system; or
the schedule is based on user input received via the MEAD system.

13. The non-transitory machine-readable storage medium of claim 11 further comprising:
receiving first sensor data associated with the microwave generator intermittently generating the microwave energy to destroy the at least a portion of the contaminants from airflow; and
causing, based on the first sensor data, performance of a corrective action associated with the MEAD system.

14. The non-transitory machine-readable storage medium of claim 11, wherein the operations further comprise:
receiving historical sensor data associated with one or more MEAD systems;
receiving historical performance data associated with the one or more MEAD systems; and
training a machine learning model with data input comprising the historical sensor data and target data comprising the historical performance data to generate a trained machine learning model, the trained machine learning model capable of generating one or more outputs indicative of predictive data for performing one or more corrective actions.

15. The non-transitory machine-readable storage medium of claim 13, wherein the operations further comprise:
providing the first sensor data to a trained machine learning model; and
obtaining, from the trained machine learning model, one or more outputs indicative of predictive data, wherein the causing of the performance of the corrective action is based on the predictive data.

16. A system comprising:
memory; and
a processing device coupled to the memory, wherein the processing device is to:
identify a schedule to operate a microwave enhanced air disinfection (MEAD) system; and
causing, based on the schedule, intermittent generation of microwave energy by a microwave generator of the MEAD system, wherein a multi-component filter disposed in a housing of the MEAD system is configured to collect contaminants from airflow through the housing, and wherein at least a portion of the contaminants from the airflow is to be destroyed at least one of directly or indirectly via the microwave energy.

17. The system of claim 16, wherein one or more of:
the schedule is based on sensor data associated with one or more MEAD systems;
the schedule is based on first sensor data received from a sensor of the MEAD system; or
the schedule is based on user input received via the MEAD system.

18. The system of claim 16 further comprising:
receive first sensor data associated with the microwave generator intermittently generating the microwave energy to destroy the at least a portion of the contaminants from airflow; and
cause, based on the first sensor data, performance of a corrective action associated with the MEAD system.

19. The system of claim 16, wherein the processing device is further to:
receive historical sensor data associated with one or more MEAD systems;
receive historical performance data associated with the one or more MEAD systems; and
train a machine learning model with data input comprising the historical sensor data and target data comprising the historical performance data to generate a trained machine learning model, the trained machine learning model capable of generating one or more outputs indicative of predictive data for performing one or more corrective actions.

20. The system of claim 18, wherein the processing device is further to:
provide the first sensor data to a trained machine learning model; and
obtain, from the trained machine learning model, one or more outputs indicative of predictive data, wherein the processing device is to cause the performance of the corrective action based on the predictive data.
